SEQUENCE LISTING (6 sequences)

SEQ ID NO: 1 - protein, 294 aa, Hordeum vulgare
MKATAGALLAVVATVLLRGIAAAPPRKPVDVPFEKNYVPTWAEDHIHYVNGGREVQLSLDKTTGTGFQTRGSYLFGHFSMHIKLVGGDSAGTVTAFYLSSQNSEHDEIDFEFLGNRTGQPYILQTNVFSGGKGDREQRIYLWFDPTKDYHSYSVLWNLYMIAFFVDDTPIRVFKNSKDLGVRYPFDQPMKLYSSLWNADDWATRGGREKTDWSKAPFVASYRGFHVDGCEASAEAKLCATQGARWWDQPEFQDLDAAQYRRLAWVRKEHTIYNYCTDRERYAAMSPECKRDRDV

SEQ ID NO: 2 - protein, 285 aa, Hordeum vulgare
MASGPSRTVPCSVLPLLLLLAGVARAAGNFYQDVDITWGDGRGKILGGGDLLTLSLDRASGSGFQSKNQYLYGRFDMQIKLVPGDSAGTVATFYLSSQGSAHDEIDFEFLGNASGQPYTVHTNVYSQGKGGREQQFRMWFDPTADFHTYSVLWNPTHILFYVDGTPIREHRNREAATGVPYLRSQAMRVYASVWDAEEWATQGGRVRTDWSRAPFVASYKGLAASGCASQDAAACANSNGAWMYQELDATALDRLQWVQKNYMIYNYCTDTWRFKDGAPPECASK

SEQ ID NO: 3 - protein, 301 aa, Hordeum vulgare
LDTSPVPFDAGYAPLFGGDNLVRSADGRSVTLKLDRYTGSGFVSKSAYRHGFFGASIKLPGDYTAGVVVAFYLSNWDEYPKNHDELDFELLGNRRGHGWRVQTNMYGNGSTARGREERYHLPVEPTVTGVHRYAIAWTPNNIVFYLDGVPIREVVRVPSMGGDFPSKPMSVYATIWDGSAWATDGGKYKVDYAYAPFAAEFSDLVLSGCGAGNVADPEGCQVDLLTHDVAVMAPAKRAAMRGFREQHLTYTACRDRVRYKTTVFPECDDLADGDSSFHLWGESKKRRRRSSSPLQYSSSMQ

SEQ ID NO: 4 - DNA, 882 bp, Hordeum vulgare
atgaaggctaccgcgggggccctcctcgccgtggtggccacggtgctactgcgaggcatcgcggcagcgccgccccggaagccggtggacgtgccattcgagaagaactacgtcccgacatgggcggaggaccacatccactacgtgaacggcggacgggaggtgcagctgtccctcgacaagaccaccggcactggcttccagacccggggctcctacctcttcggccacttcagcatgcacatcaagctcgtcggcggcgactccgccggcacagtcaccgccttctacctgtcgtcgcagaactcggagcacgacgagatcgacttcgagttcttggggaacaggacggggcagccgtacatcctgcagacgaacgtgttctccggcgggaagggcgaccgggaacagaggatctacctctggttcgacccaaccaaggactaccactcctactccgtcctctggaacctctacatgatcgcgttctttgtggacgacacgccgatccgggtgttcaagaacagcaaggacctcggcgtgcggtaccccttcgaccagcctatgaagctctactcgagcctgtggaacgcggacgactgggcgactcggggagggcgggagaagacggactggtccaaggcgcccttcgtcgcctcctaccggggcttccacgtcgacggctgcgaggcgtcggcggaggccaagttgtgcgccacccagggcgcccgctggtgggatcagcccgagttccaggacctggacgccgcgcagtaccgccgcctcgcctgggtcaggaaggagcacaccatctacaactactgcacagaccgcgaacgatacgccgccatgtcgcccgagtgcaaacgcgaccgcgacgtc

SEQ ID NO: 5 - DNA, 855 bp, Hordeum vulgare
atggcgtccggtcccagtagaacagtcccgtgctctgtgctgccactgctgctgctgctcgccggcgtggcccgcgcggccggcaacttctaccaggacgtggacatcacgtggggcgacgggcgcggcaagatcctcggcggcggcgacctcctcacgctgtcgctcgacagggcctccggctccgggttccagtccaagaaccagtacctgtacggccgcttcgacatgcagatcaagctcgtccccggcgactccgccggcaccgtcgccactttctacctgtcgtcgcaggggtcggcgcacgacgagatcgacttcgagttcctgggcaacgcgagcgggcagccctacacggtgcacaccaacgtgtacagccagggcaagggcggccgggagcagcagttccgcatgtggttcgaccccaccgccgacttccacacctactccgtcctctggaaccccacacacatcctgttctacgtggacgggacgccgatacgggagcaccgcaaccgggaggcggcgacgggggttccctacctgcggagccaggcgatgagggtgtacgcgagcgtgtgggacgcggaagagtgggcgacgcagggcgggcgggtgaggacggactggtcgcgggcgccgttcgtggcgtcgtacaaggggctcgccgcgagcgggtgcgcgtcgcaggacgcggcggcgtgcgccaactccaacggcgcgtggatgtaccaggagctggacgccacggcgttggaccgcctccagtgggtgcagaagaactacatgatctacaactactgcacggacacgtggaggttcaaggacggcgccccgcccgagtgcgccagcaag

SEQ ID NO: 6 - DNA, 903 bp, Hordeum vulgare
ctggacacgtcgccggtgccgttcgacgccgggtacgcgcctctcttcggcggcgacaacctcgtccggtcggcggacggccggagcgtcacgctcaagctggaccgatacaccgggtctgggttcgtgtcaaagtcggcctaccgccatggcttcttcggcgcttccattaagctgcccggcgactataccgccggcgtcgtcgtcgccttctacctatcgaactgggacgagtaccccaagaaccacgacgagctggacttcgagctgctgggcaaccggcgcggccacggctggcgtgtccagaccaacatgtacggcaacggcagcaccgcccgcggccgagaggagcgttaccatctccccgtcgaacctaccgtcaccggcgtccaccgctatgccattgcctggacccccaacaacatcgtcttctacctcgacggcgttcccatccgtgaggttgtccgcgttccctccatgggcggcgacttcccctccaagcccatgtccgtgtacgccaccatctgggacggctccgcctgggcgaccgacggcggcaagtacaaggtcgactacgcctacgcgcccttcgctgccgagttttccgacctcgtcctgagcggctgcggcgcgggcaacgtcgccgaccccgaggggtgccaggtcgacctgctcacgcacgacgtcgcagtcatggcaccggccaagcgcgccgccatgagagggttccgcgagcagcacctaacctacacggcgtgccgcgacagggtgcgatacaagaccaccgtcttccccgagtgtgacgacctcgccgacggcgactccagcttccacctctggggggagtccaagaagaggcgccgccggtcgtcctcgccgctgcagtactcttccagcatgcag

(19) United States
(12) Patent Application Publication
(10) Pub. No.: US 2010/0009414 A1
(43) Pub. Date: Jan. 14, 2010

(54) POLYSACCHARIDE TRANSFERASE

(75) Inventors: Maria Hrmova, Felixstow (AU); Geoffrey Bruce Fincher, Hazelwood Park (AU)

Correspondence Address: MCKEE, VOORHEES & SEASE, P.L.C., 801 GRAND AVENUE, SUITE 3200, DES MOINES, IA, 50309-2721, US

(73) Assignee: ADELAIDE RESEARCH AND INNOVATION PTY LTD, ADELAIDE (AU)

(21) Appl. No.: 12/375,237

(22) PCT Filed: Jul. 27, 2007
(86) PCT No.: PCT/AU07/01045

§ 371 Date: May 13, 2009

(30) Foreign Application Priority Data
Jul. 27, 2006 (AU) 2006904043

(51) Int. Cl. C12P 19/04; C12N 9/10; C07H 1/00; C12N 5/04

(52) U.S. Cl. 435/101; 435/193; 536/123.1; 435/410

(57) ABSTRACT

The present invention is predicated on the discovery that a polysaccharide transferase purified from barley seedlings can catalyze the formation of covalent bonds between different polysaccharides, including between xyloglucans and cellulose, and between xyloglucans and (1,3;1,4)-β-D-glucans. The present invention thus provides, among other things, an isolated or substantially purified polysaccharide transferase, wherein said polysaccharide transferase is capable of catalyzing the formation of a covalent bond between a donor polysaccharide and an acceptor polysaccharide; or a functionally active fragment or variant of said polysaccharide transferase.

### FIELD OF THE INVENTION

The present invention relates generally to enzymes which act on polysaccharide substrates. More particularly, the present invention provides isolated or substantially purified polysaccharide transferases.

### BACKGROUND OF THE INVENTION

This International Patent Application claims priority to Australian Provisional Patent Application 2006904903, the specification of which is hereby incorporated by reference.

Plant cell walls are dynamic structures that are altered during cell division, growth and differentiation to enable cells to adapt to changing functional requirements and to environmental and pathogen-induced challenges. The overall strength and flexibility of land plants are determined largely through the collective strength and flexibility of the walls that surround individual cells. In addition, walls are important for intercellular cohesion and cell-cell communication, and must be selectively permeable to water, nutrients and phytohormones.

The primary cell walls of vascular plants consist of cellulosic microfibrils that are embedded in a chemically complex matrix consisting mostly of polysaccharides, but also containing structural proteins and enzymes, and phenolic acids. Xyloglucans and pectic polysaccharides are the major non-cellulosic polysaccharides of primary walls from dicotyledonous plants, while in the Poales and related commelinoid monocots, including commercially important cereals and grasses, glucuronoarabinoxylans and (1,3;1,4)-β-D-glucans are the predominant non-cellulosic wall polysaccharides, and levels of pectic polysaccharides, glucomannans and xyloglucans are relatively low.

However, wall composition and the fine structures of component polysaccharides vary depending upon the growth phase, cell type, cell position, and local region within the wall. In some cell types, lignin is deposited throughout the wall during secondary thickening and, in response to pathogen attack, the rapid formation of a cross-linked protein network, together with the deposition of callose and lignin, can create a physical barrier to invading microorganisms.

As our understanding of the importance and contributions of different wall components to agro-industrial processes such as paper and pulping, food quality and texture, malting and brewing, bioethanol production, dietary fibre and ruminant digestibility grows, there are increasing attempts to apply genetic manipulation and conventional breeding techniques to modify the quality and quantity of individual components. Furthermore, it has been shown recently that stem strength in maize is closely correlated with cellulose content and alteration of cellulose content might therefore be expected to affect agronomically important properties such as lodging and digestibility.

Although the compositions of plant cell walls have been defined in detail, there is little information on the molecular interactions between constituent polysaccharides in the wall. In addition, manipulation of the major wall polysaccharides via their biosynthesis has also been hampered by a lack of knowledge of the mechanism(s) and control of the biosynthetic steps, coupled with a limited understanding of the physical and chemical interactions between wall components.

In most plant cell wall models it has been assumed that different polysaccharides are held in place through extensive intermolecular hydrogen bonding rather than through covalent bonds. However, in accordance with the present invention, a polysaccharide transferase has been isolated from a plant cell wall, which is proposed to form a covalent bond between polysaccharides.

Covalent linkages between different polysaccharides in plant cell walls might represent a widespread phenomenon that will fundamentally change the present understanding of plant cell wall biology, and how genetic manipulation and conventional breeding techniques might be applied to improve agro-industrial processes such as paper production, food quality and texture, malting and brewing, bioethanol production, dietary fibre and ruminant digestibility.

Reference to any prior art in this specification is not, and should not be taken as, an acknowledgment or any form of suggestion that this prior art forms part of the common general knowledge in any country.

### SUMMARY OF THE INVENTION

The present invention is predicated, in part, on the isolation and functional characterisation of polysaccharide transferases which are capable of catalysing the formation of a covalent bond between a donor polysaccharide and an acceptor polysaccharide.

In accordance with the present invention, it has been shown that a polysaccharide transferase purified from barley seedlings can catalyze the formation of covalent bonds between different polysaccharides, including between xyloglucans and cellulose, and between xyloglucans and (1,3;1,4)-β-D-glucans.

In a first aspect, the present invention provides an isolated or substantially purified polysaccharide transferase, wherein said polysaccharide transferase is capable of catalyzing the formation of a covalent bond between a donor polysaccharide and an acceptor polysaccharide; or a functionally active fragment or variant of said polysaccharide transferase.

In one embodiment, the polysaccharide transferase of the present invention is capable of forming a covalent bond wherein the donor polysaccharide and/or the acceptor polysaccharide is a polysaccharide other than a xyloglucan.

In further embodiments, the present invention also contemplates isolated or substantially purified polysaccharide transferases which are capable of catalyzing the formation of a covalent bond between plant cell wall polysaccharides such as, for example, celluloses, hemicelluloses, xyloglucans, (1,3;1,4)-β-D-glucans, arabinoxylans, pectins, mannans and the like.

The polysaccharide transferases of the present invention may be produced in any suitable way or isolated from any suitable source. However, in one embodiment, the polysaccharide transferase of the present invention is purified from a plant, or a part, organ, tissue or cell thereof. In yet another embodiment, the polysaccharide transferase is purified from a plant cell wall.

In a second aspect, the present invention provides a method for isolating or substantially purifying a polysaccharide transferase from a sample, the method comprising the steps of:
(i) a salt fractionation step;
(ii) an ion-exchange chromatography step;
(iii) a hydrophobic-interaction chromatography step;
(iv) a chromatofocussing chromatography step; and
(v) a size-exclusion chromatography step.

wherein a fraction having polysaccharide transferase specific activity from one step is used in a subsequent step.

In a one embodiment, the second aspect of the invention provides a method for isolating or substantially purifying a polysaccharide transferase to a homogenous form.

In a third aspect, the present invention provides a method for forming a covalent bond between a donor polysaccharide and an acceptor polysaccharide, the method comprising contacting said donor polysaccharide and said acceptor polysaccharide with either a polysaccharide transferase which is capable of catalyzing a covalent bond between said donor polysaccharide and said acceptor polysaccharide, or a functionally active fragment or variant of said polysaccharide transferase.

In a fourth aspect, the present invention provides an isolated polysaccharide of the structure:

A−B

wherein A and B are polysaccharides which are covalently linked to each other.

Polysaccharide A and polysaccharide B may be any polysaccharides. However, in one embodiment, at least one of polysaccharide A and polysaccharide B is a polysaccharide other than a xyloglucan. Therefore, in this embodiment, the polysaccharide of the present invention comprises a xyloglucan covalently bonded to another polysaccharide of a different type and/or a polysaccharide comprising two non-xyloglucan polysaccharides (which may be the same or of a different type) covalently bonded to each other.

In a fifth aspect, the present invention also provides a method for modulating the extent of covalent bonding between a donor polysaccharide and an acceptor polysaccharide in a cell; the method comprising modulating the level or activity of either a polysaccharide transferase which is capable of catalyzing a covalent bond between said donor polysaccharide and said acceptor polysaccharide, or a functionally active fragment or variant of said polysaccharide transferase, in said cell.

In one embodiment, the method of the fifth aspect of the present invention is adapted to modulating the extent of covalent bonding between a donor polysaccharide and an acceptor polysaccharide in a plant cell. In another embodiment, the method of the fifth aspect of the present invention is adapted to modulating the extent of covalent bonding between a donor polysaccharide and an acceptor polysaccharide in a plant cell wall.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to herein by a sequence identifier number (SEQ ID NO:). A summary of the sequence identifiers is provided in Table 1. A sequence listing is provided at the end of the specification.

TABLE 1
Summary of Sequence Identifiers

| | | Identifier in |
|---|---|---|
| Sequence Identifier | Sequence | sequence listing |
| SEQ ID NO: 1 | HvXTH5 amino acid sequence | 400 <1> |
| SEQ ID NO: 2 | HvXTH6 amino acid sequence | 400 <2> |
| SEQ ID NO: 3 | HvXTH8 amino acid sequence | 400 <3> |
| SEQ ID NO: 4 | HvXTH5 nucleotide sequence | 400 <1> |
| SEQ ID NO: 5 | HvXTH6 nucleotide sequence | 400 <2> |
| SEQ ID NO: 6 | HvXTH8 nucleotide sequence | 400 <3> |

### DESCRIPTION OF PREFERRED EMBODIMENTS

It is to be understood that the following description is for the purpose of describing particular embodiments only and is not intended to be limiting with respect to the above description.

As set out above, the present invention is predicated, in part, on the isolation and functional characterisation of polysaccharide transferases which are capable of catalysing the formation of a covalent bond between a donor polysaccharide and an acceptor polysaccharide.

In one embodiment, the term “polysaccharide transferase” refers to a polysaccharide transferase that does not utilize nucleotide sugar substrates.

In another embodiment, the term “polysaccharide transferase” refers to a polysaccharide transferase which is a family GH16 glycoside hydrolase.

Family GH16 glycoside hydrolases are described in Strohmeier et al. (Protein Science 13: 3200-3213, 2004) and exemplary family GH16 glycoside hydrolases are described in “the family GH16 glycoside hydrolase database” (GHDB) at http://www.ghdb.uni-stuttgart.de.

In yet another embodiment, the term “polysaccharide transferase” refers to a xyloglucan:xyloglucosyl transferase/hydrolase (XTH).

Reference herein to a “xyloglucan:xyloglucosyl transferase/hydrolase” or an “XTH” should not be considered limiting to enzymes which act exclusively on xyloglucan substrates nor limiting to enzymes which exhibit both transferase and hydrolase activity. For example, the term XTH should also be understood to include xyloglucan endotransglycosylases (XETs) which predominantly or exclusively exhibit transglycosylation activity. The barley enzymes described herein in accordance with some embodiments of the invention may be classified as XETs.

In addition, some enzymes in the XTH family act only as endohydrolases and these may also be designated XEHs. Thus, the term XTH should also be understood to include XEHs.

Xyloglucans consist of a backbone of (1,4)-β-D-glucan substituted with xylosyl, galactosyl and fucosyl residues. The molecular sizes of xyloglucans can be altered after their deposition into the cell wall and this is likely to be mediated by a class of enzymes known as xyloglucan endotransglycosylases/hydrolases (XTHs). The XTHs are abundant in the apoplastic space, they hydrolyse the (1,4)-β-D-glucan backbone of xyloglucans and, in the case of XETs, transfer the aglycone product of hydrolysis directly onto the non-reducing terminus of another xyloglucan chain.

Sequences encoding XTHs are surprisingly abundant in barley EST databases, given the relatively low levels of xyloglucans in walls of most barley tissues. There are at least 22 XTH genes in barley and about 30 in rice. In an attempt to reconcile the relatively low abundance of xyloglucans in barley cell walls against the large number of XTH genes and their high expression levels in many tissues of barley, it is postulated that some of the XTHs might be active on the more abundant matrix phase polysaccharides of barley cell walls, for example, the arabinoxylans and the (1,3;1,4)-β-D-glucans. Indeed, molecular modeling experiments based on the three-dimensional structure of an XTH from poplar suggest that the three-dimensional dispositions of amino acid residues in the substrate-binding and catalytic sites of XTHs and microbial (1,3;1,4)-β-D-glucan endohydrolases would be similar.

The plant XTHs and microbial (1,3;1,4)-β-D-glucan endohydrolases are all classified in the family GH16 group of glycoside hydrolases, although a small number of microbial XTHs are classified within families GH12 and GH5 (http://afmb.cnrs-mrs.fr/CAZY/). A role for XETs in the modification of highly abundant (1,3;1,4)-β-D-glucans and arabinoxylans in walls of the commelinoid monocots would be consistent with the abrupt increase in molecular size of heteroxylans that has been observed in suspension-cultured maize cells following the deposition of the polysaccharide into the walls.

As set out above, the present invention is predicated, in part, on polysaccharide transferases which are capable of catalysing the formation of a covalent bond between a donor polysaccharide and an acceptor polysaccharide.

As referred to herein, a “donor polysaccharide” refers to a polysaccharide which donates the energy to a transglycosylation reaction (catalysed by the polysaccharide transferase), while an “acceptor polysaccharide” is a polysaccharide which becomes covalently linked to the donor polysaccharide, through its non-reducing end, as a result of the transglycosylation reaction.

The catalytic mechanism of the polysaccharide transferases of the present invention may be described as a “disproportionation reaction”, as the degree of polymerization of both the donor and the acceptor polysaccharide are changed.

In a first aspect, the present invention provides an isolated or substantially purified polysaccharide transferase, wherein said polysaccharide transferase is capable of catalyzing the formation of a covalent bond between a donor polysaccharide and an acceptor polysaccharide; or a functionally active fragment or variant of said polysaccharide transferase.

The meaning of the term “polysaccharide” will be well known to those of skill in the art. “Types” of polysaccharide, as used herein, refers to polysaccharides which are differentiated on the basis of the backbone and/or side chains on the backbone of the polysaccharide molecule. For example, polysaccharides may be classified as different “types” of polysaccharide on the basis of the monomeric composition (eg. glucose, mannose, xylose and the like) or the linkage types (eg. β(1-4), β(1-3) and the like) in the molecular backbone of the polysaccharide molecule. Alternatively, different types of polysaccharide may be differentiated on the basis of any side chains on the backbone of the polysaccharide. For example, cellulose and xyloglucan should be considered different types of polysaccharide on the basis of differences in side chains, although both comprise a molecular backbone of β(1-4) linked glucose molecules.

The term “polysaccharide” should also be understood to include naturally occurring polysaccharides, synthetic polysaccharides and polysaccharide variants and derivatives. Examples of polysaccharide “derivatives” may be found in Liebert and Heinze (Biomacromolecules 6: 333-340, 2005) and Heinze (In S. Dimitriu (Ed.), Polysaccharides: Structural diversity and functional versatility, (2^{nd} ed.), pp. 551-590, New York: Marcel Dekker, 2004).

The term “polysaccharide” should also be understood to specifically include the various different polysaccharide types found in plant cell walls, including, for example, celluloses, hemicelluloses, xyloglucans, arabinoxylans, glucomannans, galactomannans, (1,3)-β-D-glucans, mixed linkage β-D-glucans (eg. (1,3;1,4)-β-D-glucans), and the like.

The present invention contemplates a polysaccharide transferase which is capable of catalyzing the formation of a covalent bond between any suitable donor polysaccharide and any suitable acceptor polysaccharide. However, in one embodiment, the polysaccharide transferase of the present invention is capable of forming a covalent bond wherein the donor polysaccharide and/or the acceptor polysaccharide is a polysaccharide other than a xyloglucan. Therefore, in this embodiment, the polysaccharide transferase of the present invention is capable of catalyzing the formation of a covalent bond between a xyloglucan and another polysaccharide of a different type and/or capable of catalyzing the formation of a covalent bond between two non-xyloglucan polysaccharides (which may be the same or of a different type).

As set out above, the polysaccharide transferase of the present invention may catalyse the formation of a covalent bond between the same or different types of polysaccharide. However, in one embodiment, the polysaccharide transferase of the present invention is capable of catalyzing the formation of a covalent bond between a donor polysaccharide and an acceptor polysaccharide which are of a different type.

The polysaccharide transferase of the present invention may catalyse the formation of a covalent bond between any appropriately sized donor and acceptor polysaccharides. However, in one embodiment, the donor polysaccharide comprises a backbone of at least 10 monomer units. In another embodiment, the acceptor polysaccharide comprises a backbone of at least 4 monomer units.

In one embodiment, the present invention provides an isolated or substantially purified polysaccharide transferase which is capable of forming a covalent bond between a donor polysaccharide comprising:
(i) a xyloglucan; or
(ii) a glucose polymer (other than a xyloglucan) which comprises at least one β(1-4) linkage between two glucose monomers;

and an acceptor polysaccharide comprising:
(i) a xyloglucan; or
(ii) a glucose polymer (other than a xyloglucan) which comprises at least one β(1-4) linkage between two glucose monomers.

As referred to herein, the term “glucose polymer” should be understood to include any polysaccharide which includes a glucose monomer backbone. The glucose polymers contemplated herein may also comprise glucose or non-glucose containing side chains. Furthermore, one or more of the glucose monomers comprising the backbone of the glucose polymer may be derivatised (eg. see below regarding cellulose derivatives).

In another embodiment, the donor polysaccharide comprises a glucose polymer (other than a xyloglucan) which comprises at least one β(1-4) linkage between two glucose monomers and the acceptor polysaccharide comprises a xyloglucan.

In yet another embodiment, the donor polysaccharide comprises a xyloglucan and the acceptor polysaccharide comprises a glucose polymer (other than a xyloglucan) which comprises at least one β(1-4) linkage between two glucose monomers.

In further embodiments, the glucose polymer comprises β(1-4) linkages between all of the glucose monomers in the backbone of the polysaccharide, and in one embodiment, the glucose polymer comprises cellulose, or an oligomer or derivative thereof.

Cellulose comprises a backbone of β-glucose monomers linked together through (1→4) glycosidic bonds. The hydroxyl groups of cellulose can also be partially or fully reacted with various chemicals to generate cellulose derivates. Exemplary cellulose derivatives include: cellulose esters, such as cellulose acetate and triacetate and nitrocellulose; cellulose ethers including ethylcellulose, hydroxyethyl cellulose, hydroxypropyl cellulose, carboxymethyl cellulose, hydroxypropyl methyl cellulose, hydroxyethyl methyl cellulose; acid-swollen celluloses such as sulfuric acid swollen cellulose and phosphoric acid swollen cellulose. Further examples of cellulose derivatives are described in Liebert and Heinze (2005, supra) and Heinze (2004, supra).

In alternate embodiments, the glucose polymer may comprises at least one β(1-3) linkage between two glucose monomers in addition to at least one β(1-4) linkage between two glucose monomers, and in one embodiment, the glucose polymer comprises a 1,3;1,4-β-D-glucan or an oligomer or derivative thereof.

(1,3;1,4)-β-D-Glucans should be understood to include linear, unbranched polysaccharides in which β-D-glucopyranosyl monomers are polymerized through a mixture of both (1-4)- and (1-3)-linkages.

In further embodiments, the present invention also contemplates polysaccharide transferases which are capable of catalyzing the formation of a covalent bond between other polysaccharides. In yet further embodiments, the present invention also contemplates isolated or substantially purified polysaccharide transferases which are capable of catalyzing the formation of a covalent bond between other plant cell wall polysaccharides such as, for example, cellulosic and non-cellulosic polysaccharides (eg. xyloglycans, mannoglycans, xyloglucans, glucans) that contain β-D-glycosyl monomers polymerized through (1→4)- and (1→3)-linkages, arabinoxylans, pectins, mannans and the like.

In particular, it has been recognized that arabinoxylans comprise a backbone of 1,4-beta-linked xylosyl residues, which is similar in overall structure to cellulose (which comprises a backbone of 1,4-beta-linked glucosyl residues). The backbone of the arabinoxylan further comprises mostly single substituents of arabinose that interfere with alignment of the backbones and hence solubilise the polysaccharide. The present inventors have further recognized that arabinoxylan and xyloglucan may be functionally equivalent with respect to their structures and properties in a plant cell wall and, accordingly, arabinoxylans may also serve as donor and/or acceptor polysaccharides for the polysaccharide transferases of the present invention.

Accordingly, in another embodiment, the present invention provides an isolated or substantially purified polysaccharide transferase which is capable of forming a covalent bond between a donor polysaccharide and an acceptor polysaccharide wherein at least one of the donor and/or acceptor polysaccharide comprises an arabinoxylan.

As set out above, the present invention contemplates any polysaccharide transferase which is capable of forming a covalent bond between a donor polysaccharide and an acceptor polysaccharide. As such, the isolated or substantially purified polysaccharide transferases contemplated by the present invention may comprise any suitable amino acid sequence.

However, in one embodiment, the polysaccharide transferase comprises:
(i) the amino acid sequence set forth in SEQ ID NO: 1; or
(ii) an amino acid sequence which encodes a functionally active fragment or variant of the polysaccharide transferase referred to at (i).

In another embodiment, the polysaccharide transferase comprises:
(i) the amino acid sequence set forth in SEQ ID NO: 2; or
(ii) an amino acid sequence which encodes a functionally active fragment or variant of the polysaccharide transferase referred to at (i).

In yet another embodiment, the polysaccharide transferase comprises:
(i) the amino acid sequence set forth in SEQ ID NO: 3; or
(ii) an amino acid sequence which encodes a functionally active fragment or variant of the polysaccharide transferase referred to at (i).

As set out above, the present invention also contemplates “functionally active fragments or variants” of polysaccharide transferases comprising the amino acid sequence set forth in any of SEQ ID NO: 1, SEQ ID NO: 2 and SEQ ID NO: 3.

“Functionally active fragments”, as contemplated herein, may be of any length wherein the fragment retains the capability to catalyse a covalent bond between a donor and acceptor polysaccharide. The fragment may comprise at least 100 amino acid residues, at least 150 amino acid residues, at least 200 amino acid residues or at least 250 amino acid residues. For example, in one embodiment, a fragment at least 100 amino acid residues in length comprises fragments which include 100 or more contiguous amino acids from, for example, the amino acid sequence of any of SEQ ID NO: 1, SEQ ID NO: 2 or SEQ ID NO: 3.

“Functionally active variants” of the polysaccharide transferases of the invention include orthologs, mutants, synthetic variants, analogs and the like which retain the capability to catalyse the formation of a covalent bond between a donor polysaccharide and an acceptor polysaccharide. For example, the term “variant” should be considered to specifically include, for example, orthologous polysaccharide transferases derived from different organisms; naturally occurring or synthetic mutants of the polysaccharide transferase; variants of the polysaccharide transferase wherein one or more amino acids within the sequence has been substituted, added or deleted; analogs that contain one or more modified amino acids modified for stability or for other reasons; and chemically synthesised forms of the polysaccharide transferase.

In some embodiments, the functionally active fragment or variant comprises at least 30% sequence identity, at least 45% sequence identity, at least 60% sequence identity, at least 70% sequence identity, at least 80% sequence identity, at least 85% sequence identity, at least 90% sequence identity, at least 92% sequence identity, at least 94% sequence identity, at least 96% sequence identity, at least 97% sequence identity, at least 98% sequence identity, or at least 99% sequence identity to the amino acid sequence set forth in any of SEQ ID NO: 1, SEQ ID NO: 2 or SEQ ID NO: 3.

When comparing amino acid sequences to calculate a percentage identity, the compared amino acid sequences should be compared over a comparison window of at least 20 amino acid residues, at least 50 amino acid residues, at least 100 amino acid residues, at least 200 amino acid residues, at least 250 amino acid residues, or over the full length of any of SEQ ID NO: 1, SEQ ID NO: 2 or SEQ ID NO: 3. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19.3 of Ausubel et al. (“Current Protocols in Molecular Biology” John Wiley & Sons Inc, 1994-1998, Chapter 15, 1998).

In another embodiment, the polysaccharide transferase of the present invention is glycosylated, or otherwise modified by post-translational processes.

The meaning of the term “glycosylated” would be readily determined by one of skill in the art and should be understood to specifically include, among other things, the post-translational attachment of N-linked and O-linked oligosaccharides to the polysaccharide transferase protein. As referred to herein, “other” post-translational modifications should be understood to refer to post-translational modifications such as, for example, phosphorylation, proteolytic cleavage, acylation, methylation, sulphation, prenylation, selenocysteine incorporation and the like.

In one embodiment, the polysaccharide transferase of the present invention comprises a plant-type glycosylation or other post-translational modification pattern, or a functionally equivalent glycosylation or other post-translational modification pattern.

As referred to herein, a polysaccharide transferase comprising a “plant-type glycosylation or other post-translational modification pattern” refers to the polysaccharide transferase having a glycosylation pattern or other post-translational modification pattern that is the same as, or functionally equivalent to, the glycosylation or other post-translational modification pattern that is used by plants. In one embodiment, a polysaccharide transferase comprising a “plant-type glycosylation or other post-translational modification pattern” refers to the polysaccharide transferase having the same glycosylation pattern or other post-translational modification pattern, or a functional equivalent thereof, of a polysaccharide transferase isolated from a plant.

The polysaccharide transferases of the present invention may be produced in any suitable way or isolated from any suitable source. However, in one embodiment, the polysaccharide transferase of the present invention is purified from a plant, or a part, organ, tissue or cell thereof. In another embodiment, the polysaccharide transferase is purified from a plant cell wall.

The present invention contemplates the purification of polysaccharide transferases from any plant, plant cell or plant cell wall. As such, in some embodiments, the present invention contemplates the purification of polysaccharide transferases from monocotyledonous angiosperm plants, dicotyledonous angiosperm plants and gymnosperm plants.

In one embodiment, however, the present invention provides a polysaccharide transferase isolated from a monocotyledonous plant. In another embodiment, the present invention provides a polysaccharide transferase purified from a cereal crop plant or other member of the Poaceae.

As used herein, the term “cereal crop plant” includes members of the order Poales and/or the family Poaceae, which produce edible grain for human or animal food. Examples of cereal crop plants that in no way limit the present invention include barley, wheat, rice, maize, millets, sorghum, rye, triticale, oats, teff, rice, spelt and the like. However, the term “cereal crop plant” should also be understood to include a number of non-Poales species that also produce edible grain, which are known as pseudocereals, and include, for example, amaranth, buckwheat and quinoa.

In one embodiment, the polysaccharide transferase of the present invention is purified from a barley (Hordeum vulgare) plant or a cell or cell wall thereof.

The present invention also contemplates an isolated or substantially purified polysaccharide transferase produced in a recombinant expression system.

A vast array of recombinant expression systems that may be used to express a polysaccharide transferase-encoding a nucleic acid are known in the art. Exemplary “recombinant expression systems” include: bacterial expression systems such as E. coli expression systems (reviewed in Baneyx, Curr. Opin. Biotechnol. 10: 411-421, 1999; eg. see also Gene expression in recombinant microorganisms, Smith (Ed.), Marcel Dekker, Inc. New York, 1994; and Protein Expression Technologies: Current Status and Future Trends, Baneyx (Ed.), Chapters 2 and 3, Horizon Bioscience, Norwich, UK, 2004), Bacillus spp. expression systems (eg. see Protein Expression Technologies: Current Status and Future Trends, supra, chapter 4) and Streptomyces spp. expression systems (eg. see Practical Streptomyces Genetics, Kieser et al., (Eds.), Chapter 17, John Innes Foundation, Norwich, UK, 2000); fungal expression systems including yeast expression systems such as Saccharomyces spp., Schizosaccharomyces pombe, Hansenula polymorpha and Pichia spp. expression systems and filamentous fungi expression systems (eg. see Protein Expression Technologies: Current Status and Future Trends, supra, chapters 5, 6 and 7; Buckholz and Gleeson, Bio/Technology 9(11): 1067-1072, 1991; Cregg et al., Mol. Biotechnol. 16(1): 23-52, 2000; Cereghino and Cregg, FEMS Microbiology Reviews 24: 45-66, 2000; Cregg et al., Bio/Technology 11: 905-910, 1993); mammalian cell expression systems including Chinese Hamster Ovary (CHO) cell based expression systems (eg. see Protein Expression Technologies: Current Status and Future Trends, supra, chapter 9); insect cell cultures including baculovirus expression systems (eg. see Protein Expression Technologies: Current Status and Future Trends, supra, chapter 8; Kost and Condreay, Curr. Opin. Biotechnol. 10: 428-433, 1999; Baculovirus Expression Vectors: A Laboratory Manual WH Freeman & Co., New York, 1992; and The Baculovirus Expression System: A Laboratory Manual, Chapman & Hall, London, 1992); Plant cell expression systems such as tobacco, soybean, rice and tomato cell expression systems (eg. see review of Hellwig et al., Nat Biotechnol 22: 1415-1422, 2004); cell-free expression systems (eg. see review of Katzen et al. Trends Biotechnol. 23(3): 150-156, 2005) and the like.

As set out above, the present invention provides an “isolated” or “substantially purified” polysaccharide transferase. As referred to herein, “isolated” refers to the polysaccharide transferase being removed from its original environment (e.g., the natural environment if it is naturally occurring), and thus is altered “by the hand of man” from its natural state.

“Substantially purified” polysaccharide transferases may be either pure or part of a mixture. When part of a mixture, the polysaccharide transferase activity is generally higher in the mixture than any other enzymatic activity in the mixture.

In one embodiment, however, the isolated or substantially purified polysaccharide transferase of the present invention is purified to a substantially homogenous form.

The polysaccharide transferases of the present invention may be isolated or purified from any source using any suitable method.

However, in a second aspect, the present invention provides a method for isolating or substantially purifying a polysaccharide transferase from a sample, the method comprising the steps of:
(i) a salt fractionation step;
(ii) an ion-exchange chromatography step;
(iii) a hydrophobic-interaction chromatography step;
(iv) a chromatofocussing chromatography step; and
(v) a size-exclusion chromatography step.

wherein a fraction having polysaccharide transferase specific activity from one step is used in a subsequent step.

The “sample” contemplated for use in the method of the second aspect of the invention may be any sample comprising a polysaccharide transferase protein. In one embodiment the sample is of biological origin, and in another embodiment, the sample comprises one or more cells, or a homogenate thereof. In yet another embodiment, the sample comprises any of one or more plant cells, a homogenate thereof, or one or more plant cell walls.

In one embodiment, the salt fractionation step comprises an ammonium sulphate fractionation step. In further embodiments, the ammonium sulphate fractionation step comprises fractionation using ammonium sulphate at a concentration of at least 50%, a concentration of at least 65%, a concentration of at least 80% or a concentration of about 90%.

In one embodiment, the ion-exchange chromatography step comprises an anion exchange chromatography step. In another embodiment the anion exchange chromatography step comprises anion exchange using a stationary phase comprising Sepharose Q anion exchange resin. In further embodiments, the anion exchange step is performed at a mildly acidic pH, at a pH of between 6.0 and 7.0, at a pH of between 6.5 and 7.0 or at a pH of about 6.8.

In another embodiment, the hydrophobic interaction chromatography step comprises hydrophobic interaction chromatography using a stationary phase comprising non-polar groups, such as phenyl non-polar groups. In another embodiment, the stationary phase comprises phenyl Sepharose. In further embodiments, the hydrophobic interaction chromatography step is performed at a mildly acidic pH, at a pH of between 5.0 and 7.0, at a pH of between 5.5 and 6.5 or at a pH of about 6.0.

In another embodiment, the chromatofocussing step comprises using a PBE-94 ion exchange resin. In another embodiment, the chromatofocussing step comprises elution in a pH range of about 5.0 to about 8.3.

In another embodiment, the size exclusion chromatography step comprises using a porous polyacrylamide resin. In another embodiment, the size exclusion chromatography step comprises a fractionation range of about 3000 to about 60000 Daltons. In yet another embodiment, the size exclusion chromatography step comprises using a Bio-Gel P-60 stationary phase. In a further embodiment, the size exclusion chromatography step is performed at substantially neutral pH.

In one embodiment, the second aspect of the invention provides a method for isolating or substantially purifying a polysaccharide transferase to a homogenous form.

In a third aspect, the present invention provides a method for forming a covalent bond between a donor polysaccharide and an acceptor polysaccharide, the method comprising contacting said donor polysaccharide and said acceptor polysaccharide with either a polysaccharide transferase which is capable of catalyzing a covalent bond between said donor polysaccharide and said acceptor polysaccharide, or a functionally active fragment or variant of said polysaccharide transferase.

In some embodiments, the donor and/or acceptor polysaccharides used in accordance with the method of the third aspect of the invention are as described above in connection with the first aspect of the invention.

In further embodiments, the polysaccharide transferase used in accordance with the method of the third aspect of the invention is as described above in connection with the first aspect of the invention.

The method of the third aspect of the invention may be applied to the formation of a covalent bond between a donor polysaccharide and an acceptor polysaccharide under any suitable conditions. However, in one embodiment, the method of the third aspect of the invention is adapted to forming the covalent bond in vitro.

As would be recognized by one of skill in the art, the polysaccharide transferases of the present invention have broad-ranging utility. For example, labelled polysaccharides (either radioactively labelled or labelled by introducing fluorescent tags onto their reducing termini) are required for investigating the mode of action of hydrolytic and other types of enzymes and their action patterns. These fluorescent tags could be introduced onto different types of polysaccharides by various types of polysaccharide transferases described herein.

In a fourth aspect, the present invention provides an isolated polysaccharide of the structure:

A−B

wherein A and B are polysaccharides which are covalently linked to each other.

Polysaccharide A and polysaccharide B may be any polysaccharides. However, in one embodiment, at least one of polysaccharide A and polysaccharide B is a polysaccharide other than a xyloglucan. Therefore, in this embodiment, the polysaccharide of the present invention comprises a xyloglucan covalently bonded to another polysaccharide of a different type and/or a polysaccharide comprising two non-xyloglucan polysaccharides (which may be the same or of a different type) covalently bonded to each other.

Polysaccharides A and B in the polysaccharide of the present invention may be of any size. However, in one embodiment polysaccharide A comprises a backbone of at least 10 monomer units and polysaccharide B comprises a backbone of at least 4 monomer units. In another embodiment, polysaccharide A comprises a backbone of at least 4 monomer units and polysaccharide B comprises a backbone of at least 10 monomer units.

In one embodiment, polysaccharide A comprises:
(i) a xyloglucan; or
(ii) a glucose polymer (other than a xyloglucan) which comprises at least one β(1-4) linkage between two glucose monomers

and polysaccharide B comprises:
(i) a xyloglucan; or
(ii) a glucose polymer (other than a xyloglucan) which comprises at least one β(1-4) linkage between two glucose monomers

In further embodiments, polysaccharide A comprises a glucose polymer (other than a xyloglucan) which comprises at least one β(1-4) linkage between two glucose monomers and polysaccharide B comprises a xyloglucan, or polysaccharide A comprises a xyloglucan and polysaccharide B comprises a glucose polymer (other than a xyloglucan) which comprises at least one β(1-4) linkage between two glucose monomers.

In yet further embodiments, the glucose polymer comprises β(1-4) linkages between all of the glucose monomers in the backbone of the polysaccharide and in one embodiment, the glucose polymer comprises cellulose, or an oligomer or derivative thereof.

In an alternate embodiment, the glucose polymer may comprises at least one β(1-3) linkage between two glucose monomers in addition to at least one β(1-4) linkage between two glucose monomers and in another embodiment, the glucose polymer comprises a 1,3;1,4-β-D-glucan or an oligomer or derivative thereof.

In further embodiments, the present invention also contemplates polysaccharides A and/or B being other polysaccharides, particularly plant cell wall polysaccharides such as, for example, arabinoxylans, pectins, mannans and the like.

In a yet further embodiment, the polysaccharide of the fourth aspect of the invention is produced according to the method of the third aspect of the invention.

In a fifth aspect, the present invention also provides a method for modulating the extent of covalent bonding between a donor polysaccharide and an acceptor polysaccharide in a cell; the method comprising modulating the level or activity of either a polysaccharide transferase which is capable of catalyzing a covalent bond between said donor polysaccharide and said acceptor polysaccharide, or a functionally active fragment or variant of said polysaccharide transferase, in said cell.

In some embodiments, the donor and/or acceptor polysaccharides used in accordance with the method of the fifth aspect of the invention are as described above in connection with the first aspect of the invention.

In further embodiments, the polysaccharide transferase used in accordance with the method of the fifth aspect of the present invention is as described above with regard to the first aspect of the invention.

In one embodiment, the method of the fifth aspect of the present invention is adapted to modulating the extent of covalent bonding between a donor polysaccharide and an acceptor polysaccharide in a plant cell. In another embodiment, the method of the fifth aspect of the present invention is adapted to modulating the extent of covalent bonding between a donor polysaccharide and an acceptor polysaccharide in a plant cell wall.

The chemistry and enzymology of interactions between polysaccharides and other components in the wall are crucial determinants of overall plant strength, wall porosity, and susceptibility of the wall to enzymatic degradation. The discovery of the activity of polysaccharide transferases to covalently cross-linking wall polysaccharides will enable the number of such linkages to be manipulated to either increase or decrease strength, porosity and susceptibility to pathogen attack. Thus, modulation of the extent of covalent bonding between plant cell wall polysaccharides, particularly cereal cell wall polysaccharides, has the potential to enhance cereal quality and industrial value.

One particularly promising agro-industrial application is in the replacement of fossil fuels with bioethanol. Currently, bioethanol production is increasing rapidly, and with recent advances in the catalytic efficiency of hydrolytic enzymes, it is becoming apparent that ligno-cellulosic complexes and other crop residues that consist predominantly of cellulose and non-cellulosic polysaccharides of wall origin, will become economical as a source of fermentable sugars for ethanol production. Cereal straws and corn stover are abundant, renewable sources of cellulose for ethanol production. Attention so far has been focused on the enzymatic degradation of the cellulose and non-cellulosic polysaccharides, but increased knowledge of biosynthetic mechanisms will also provide opportunities to manipulate the fine structures of the polysaccharides, the interactions of cellulose and non-cellulosic polysaccharides within the wall, and their relative abundance in walls during plant growth, such that the crop residues will be more amenable to rapid enzymatic degradation and the products of hydrolysis could be modified to enhance the efficiency of the fermentation process.

In further examples, decreased polysaccharide cross-linking in plant cell walls could be induced late in the development or during senescence of cereal straws, corn stover, sugarcane bagasse, etc. to enhance the digestibility of these residues and grasses more generally in animals, and to facilitate industrial processes such as pulp and paper manufacture.

In a yet further example, increased polysaccharide cross-linking in plant cell walls could be used to increase stem strength and hence to reduce crop losses due to lodging.

The method of the fifth aspect of the present invention may be applied to modulating the extent of covalent bonding between a donor polysaccharide and an acceptor polysaccharide in any plant cell or cell wall thereof, including, for example, monocotyledonous angiosperm plant cells, dicotyledonous angiosperm plant cells and gymnosperm plant cells.

In one embodiment, however, the plant cell is a monocotyledonous plant cell and in another embodiment the plant is a cereal crop plant cell or other member of the Poaceae family.

As referred to herein, the “modulating the extent of covalent bonding between a donor polysaccharide and an acceptor polysaccharide in a cell” should be understood to include an increase or decrease in the extent of covalent bonding between a donor polysaccharide and an acceptor polysaccharide in the cell or cell wall thereof, relative to the wild type of the cell.

As set out above, modulation of the extent of covalent bonding between a donor polysaccharide and an acceptor polysaccharide in a cell is effected by modulating the level or activity of either a polysaccharide transferase which is capable of catalyzing a covalent bond between said donor polysaccharide and said acceptor polysaccharide, or a functionally active fragment or variant of said polysaccharide transferase, in said cell.

Modulation of the “level” of the polysaccharide transferase or functionally active fragment or variant thereof should be understood to include modulation of the level of polysaccharide transferase or functionally active fragment or variant thereof transcripts and/or polypeptides in the cell. Modulation of the “activity” of the polysaccharide transferase or functionally active fragment or variant thereof should be understood to include modulation of the total activity, specific activity, half-life and/or stability of the polysaccharide transferase or functionally active fragment or variant thereof in the cell.

By “modulating” with regard to the level and/or activity of the polysaccharide transferase or functionally active fragment or variant thereof includes decreasing or increasing the level and/or activity of polysaccharide transferase or functionally active fragment or variant thereof in the cell. By “decreasing” is intended, for example, at least a 1%, 5%, 10%, 20%, 30%, 40%, 50%, 60%, 70%, 80%, 90%, 91%, 92%, 93%, 94%, 95%, 96%, 97%, 98%, 99%, 100%, reduction in the level or activity of polysaccharide transferase or functionally active fragment or variant thereof in the cell. By “increasing” is intended, for example, at least a 1%, 5%, 10%, 20%, 30%, 40%, 50%, 60%, 70%, 80%, 90%, 100%, 2-fold, 5-fold, 10-fold, 20 fold, 50-fold, 100-fold increase in the level of activity of polysaccharide transferase or functionally active fragment or variant thereof in the cell.

“Modulating” also includes introducing a polysaccharide transferase or functionally active fragment or variant thereof into a cell which does not normally express the introduced enzyme, or the substantially complete inhibition of polysaccharide transferase or functionally active fragment or variant thereof activity in a cell that normally has such activity.

In one embodiment, the extent of covalent bonding between a donor polysaccharide and an acceptor polysaccharide in a cell is increased by increasing the level and/or activity of a polysaccharide transferase or functionally active fragment or variant thereof in the cell. In another embodiment, the extent of covalent bonding between a donor polysaccharide and an acceptor polysaccharide in a cell is decreased by decreasing the level and/or activity of a polysaccharide transferase or functionally active fragment or variant thereof in the cell.

The method of the fifth aspect of the invention contemplates any means known in the art by which the level and/or activity of a polysaccharide transferase or functionally active fragment or variant thereof may be modulated in a cell. This includes, for example:
(i) modulating the expression of a nucleic acid which encodes a polysaccharide transferase or functionally active fragment or variant thereof in the cell;
(ii) the application of agents which modulate the activity of a polysaccharide transferase or functionally active fragment or variant thereof in the cell, including the application of a polysaccharide transferase agonist or antagonist;
(iii) the application of agents which mimic polysaccharide transferase activity in a cell; or
(iv) effecting the expression of an altered or mutant polysaccharide transferase encoding nucleic acid in a cell such that a polysaccharide transferase with increased or decreased specific activity, half-life and/or stability is expressed by the cell.

In one embodiment, the level and/or activity of a polysaccharide transferase or functionally active fragment or variant thereof is modulated by modulating the expression of a nucleic acid which encodes a polysaccharide transferase or functionally active fragment or variant thereof (referred to hereafter as ‘a polysaccharide transferase-encoding nucleic acid’) in the cell.

The term “modulating” with regard to the expression of a polysaccharide transferase-encoding nucleic acid is intended decreasing or increasing the transcription and/or translation of the nucleic acid. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100%, reduction in transcription and/or translation. By “increasing” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in transcription and/or translation. Modulating also comprises introducing expression of a nucleic acid not normally found in a particular cell; or the substantially complete inhibition (eg. knockout) of expression in a cell that normally has such activity.

Methods for modulating the expression of a particular nucleic acid molecule in a cell are known in the art, and the present invention contemplates any such method.

In one embodiment, the expression of a polysaccharide transferase-encoding nucleic acid is modulated by genetic modification of the cell. Exemplary types of genetic modification include, for example:
(i) random mutagenesis such as transposon, chemical, UV and phage mutagenesis together with selection of mutants which overexpress or underexpress an endogenous polysaccharide transferase-encoding nucleic acid;
(ii) transient or stable expression of one or more introduced nucleic acid molecules into a cell which direct the expression and/or overexpression of polysaccharide transferase-encoding nucleic acid in the cell;
(iii) overexpression of a polysaccharide transferase-encoding nucleic acid in a cell via transient or stable expression of additional copies of a polysaccharide transferase-encoding nucleic acid in the cell; or transient or stable expression of a polysaccharide transferase-encoding nucleic acid operably connected to a transcriptional control sequence that effects greater than wild-type expression of a polysaccharide transferase-encoding nucleic acid in the cell (for examples of plant transformation and expression of an introduced nucleotide sequence see Zhao et al. (Mol Breeding DOI 10.1007/s11032-006-9005-6, 2006), Katsuhara et al. (Plant Cell Physiol 44(12): 1378-1383, 2003), Ohta et al. (FEBS Letters 532: 279-282, 2002) and Wu et al. (Plant Science 169: 65-73, 2005);
(iv) insertional mutagenesis of a polysaccharide transferase-encoding nucleic acid in a cell including knockout or knockdown of a polysaccharide transferase-encoding nucleic acid in a cell by homologous recombination with a knockout construct (for an example of targeted gene disruption in plants see Terada et al., Nat. Biotechnol. 20: 1030-1034, 2002);
(v) post-transcriptional gene silencing (PTGS) or RNAi of a polysaccharide transferase-encoding nucleic acid in a cell (for review of PTGS and RNAi see Sharp, Genes Dev. 15(5): 485-490, 2001; and Hannon, Nature 418: 244-51, 2002);
(vi) transformation of a cell with an antisense construct directed against a polysaccharide transferase-encoding nucleic acid (for examples of antisense suppression in plants see van der Krol et al., Nature 333: 866-869; van der Krol et al., BioTechniques 6: 958-967; and van der Krol et al., Gen. Genet. 220: 204-212);
(vii) transformation of a cell with a co-suppression construct directed against a polysaccharide transferase-encoding nucleic acid (for an example of co-suppression in plants see van der Krol et al., Plant Cell 2(4): 291-299);
(viii) transformation of a cell with a construct encoding a double stranded RNA directed against a polysaccharide transferase-encoding nucleic acid (for an example of dsRNA mediated gene silencing see Waterhouse et al., Proc. Natl. Acad. Sci. USA 95: 13959-13964, 1998); and
(ix) transformation of a cell with a construct encoding an siRNA or hairpin RNA directed against a polysaccharide transferase-encoding nucleic acid (for an example of siRNA or hairpin RNA mediated gene silencing in plants see Lu et al., Nucl. Acids Res. 32(21): e171; doi:10.1093/nar/gnh170, 2004).

The present invention also contemplates the downregulation of a polysaccharide transferase-encoding nucleic acid in a cell via the use of synthetic oligonucleotides, for example, siRNAs or microRNAs directed against a polysaccharide transferase-encoding nucleic acid (for examples of synthetic siRNA mediated silencing see Caplen et al., Proc. Natl. Acad. Sci. USA 98: 9742-9747, 2001; Elbashir et al., Genes Dev. 15: 188-200, 2001; Elbashir et al., Nature 411: 494-498, 2001; Elbashir et al., EMBO J. 20: 6877-6888, 2001; and Elbashir et al., Methods 26: 199-213, 2002).

In addition to the examples above, an introduced nucleic acid may also comprise a nucleotide sequence which is not directly related to a polysaccharide transferase-encoding nucleic acid but, nonetheless, may directly or indirectly modulate the expression of a polysaccharide transferase-encoding nucleic acid in a cell. Examples include nucleic acid molecules that encode transcription factors or other proteins which promote or suppress the expression of an endogenous polysaccharide transferase-encoding nucleic acid molecule in a cell; and other non-translated RNAs which directly or indirectly promote or suppress endogenous polysaccharide transferase expression and the like.

As set out above, the method of the fifth aspect of the invention may involve transformation of a plant. Plants may be transformed using any method known in the art that is appropriate for the particular plant species. Common methods include Agrobacterium-mediated transformation, microprojectile bombardment based transformation methods and direct DNA uptake based methods. Roa-Rodriguez et al. (Agrobacterium-mediated transformation of plants, 3^{rd} Ed. CAMBIA Intellectual Property Resource, Canberra, Australia, 2003) review a wide array of suitable Agrobacterium-mediated plant transformation methods for a wide range of plant species. Other bacterial-mediated plant transformation methods may also be utilized, for example, see Broothaerts et al. (Nature 433: 629-633, 2005). Microprojectile bombardment may also be used to transform plant tissue and methods for the transformation of plants, particularly cereal plants, and such methods are reviewed by Casas et al. (Plant Breeding Rev. 13: 235-264, 1995). Direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide- and liposome mediated transformation. Methods such as these are reviewed by Rakoczy-Trojanowska (Cell. Mol. Biol. Lett. 7: 849-858, 2002). A range of other plant transformation methods may also be evident to those of skill in the art and, accordingly, the present invention should not be considered in any way limited to the particular plant transformation methods exemplified above.

Furthermore, in order to effect expression of an introduced nucleic acid in a genetically modified cell, where appropriate, the introduced nucleic acid may be operably connected to one or more transcriptional control sequences, such as a promoter.

A promoter may regulate the expression of an operably connected nucleotide sequence constitutively, or differentially, with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include, for example, a constitutive promoter, an inducible promoter, a tissue-specific promoter or an activatable promoter.

In one embodiment, a promoter or other transcriptional control sequence derived from a native polysaccharide transferase-encoding nucleic acid may be used.

The present invention also contemplates the use of any promoter which is active in a plant. Accordingly, plant-active constitutive, inducible, tissue-specific or activatable promoters may be used.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 195 (CaMV 35S and CaMV 19S) promoters; bacterial plant pathogen derived promoters such as opine promoters derived from Agrobacterium spp., eg. the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637 339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447858, Australian Patent 732872, Canadian Patent Application 1324097); cold inducible promoters (eg. see U.S. Pat. No. 6,479,260, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter may be either constitutive or inducible.

Examples of plant tissue specific promoters include: root specific promoters such as those described in US Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least an RNA polymerase binding site and, optionally a TATA box and transcription initiation site and/or one or more CAAT boxes. In one embodiment wherein the cell is a plant cell, the minimal promoter may be derived from the Cauliflower Mosaic Virus 355 (CaMV 35S) promoter. The CaMV 35S derived minimal promoter may comprise, for example, a sequence that substantially corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem 278: 248-254, 2000); Gaf1 (Hoe et al., Gene 215(2): 319-328, 1998); E2F (Albani et al., J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al., Nucl Acids Res 28: 4291-4298, 2000); MafA (Kataoka et al., J Biol Chem 277: 49903-49910, 2002); human activating transcription factor 4 (Liang and Hai, J Biol Chem 272: 24088-24095, 1997); Bcl10 (Liu et al., Biochem Biophys Res Comm 320(1): 1-6, 2004); CREB-H (Omori et al., Nucl Acids Res 29: 2154-2162, 2001); ARR1 and ARR2 (Sakai et al., Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al., Nat Genet 26: 484-489, 2000).

In one embodiment, the UAS comprises a nucleotide sequence that is able to bind to at least the DNA-binding domain of the GAL4 transcriptional activator. UAS sequences, which can bind transcriptional activators that comprise at least the GAL4 DNA binding domain, are referred to herein as UASG. In another embodiment, the UASG comprises the sequence 5′-CGGAGTACTGTCCTCCGAG-3′ or a functional homolog thereof.

As referred to herein, a “functional homolog” of the UASG sequence should be understood to refer to any nucleotide sequence which can bind at least the GAL4 DNA binding domain and which may comprise a nucleotide sequence having at least 50% identity, at least 65% identity, at least 80% identity or at least 90% identity with the UASG nucleotide sequence.

The UAS sequence in the activatable promoter may comprise a plurality of tandem repeats of a DNA binding domain target sequence. For example, in its native state, UASG comprises four tandem repeats of the DNA binding domain target sequence. As such, the term “plurality” as used herein with regard to the number of tandem repeats of a DNA binding domain target sequence should be understood to include, for example, at least 2 tandem repeats, at least 3 tandem repeats or at least 4 tandem repeats.

As set out above, the fifth aspect of the present invention contemplates “a nucleic acid which encodes a polysaccharide transferase or functionally active fragment or variant thereof” also referred to as “a polysaccharide transferase-encoding nucleic acid”. Nucleic acid sequences which encode a particular polysaccharide transferase will be readily determined by one of skill in the art. For example, the amino acid sequence of a particular polysaccharide transferase or functionally active fragment or variant thereof may be used to identify a suitable encoding nucleic acid from nucleotide sequence data such as, for example, genomic nucleotide sequence data and/or Expressed Sequence Tag (EST) data. From this, a nucleic acid which encodes a polysaccharide transferase or functionally active fragment or variant thereof may be identified and/or isolated from an organism. In another example, an amino acid sequence from a polysaccharide transferase or functionally active fragment or variant thereof may be used to determine a corresponding nucleotide sequence which may then be chemically synthesized. As would be appreciated by one of skill in the art, the codon usage in the synthetic nucleic acid may be adapted to the particular cell type into which the nucleic acid is to be introduced.

In some embodiments, the fifth aspect of the invention contemplates modulating the extent of covalent bonding between a donor polysaccharide and an acceptor polysaccharide in a cell by modulating the expression of a nucleic acid which encodes a polysaccharide transferase comprising any of:
(i) the amino acid sequence set forth in SEQ ID NO: 1;
(ii) the amino acid sequence set forth in SEQ ID NO: 2;
(iii) the amino acid sequence set forth in SEQ ID NO: 3;
(iv) an amino acid sequence which encodes a functionally active fragment or variant of the polysaccharide transferase referred to at (i), (ii) or (iii).

In another embodiment, the nucleic acid which encodes a polysaccharide transferase comprising the amino acid sequence set forth in SEQ ID NO: 1, comprises the nucleotide sequence set forth in SEQ ID NO: 4.

In another embodiment, the nucleic acid which encodes a polysaccharide transferase comprising the amino acid sequence set forth in SEQ ID NO: 2, comprises the nucleotide sequence set forth in SEQ ID NO: 5.

In another embodiment, the nucleic acid which encodes a polysaccharide transferase comprising the amino acid sequence set forth in SEQ ID NO: 3, comprises the nucleotide sequence set forth in SEQ ID NO: 6.

The isolated or substantially purified polysaccharide transferases of the present invention may also be useful, for example, in the generation of antibodies that bind to the polysaccharide transferase polypeptides.

Accordingly, in a sixth aspect, the present invention provides an antibody or an epitope binding fragment thereof, raised against a polysaccharide transferase polypeptide as defined in the first aspect of the invention.

The term “antibody”, as used herein, refers to immunoglobulin molecules and immunologically active portions of immunoglobulin molecules, i.e., molecules that contain an antigen-binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Thus, the antibodies of the present invention may include, for example: polyclonal, monoclonal, multispecific, chimeric antibodies, single chain antibodies, Fab fragments, F(ab′) fragments, fragments produced by a Fab expression library and epitope-binding fragments of any of the above.

The term “antibody”, as used herein, should also be understood to encompass derivatives that are modified, eg. by the covalent attachment of any type of molecule to the antibody such that covalent attachment does not prevent the antibody from binding to a polysaccharide transferase polypeptide or an epitope thereof. For example, the antibody derivatives include antibodies that have been modified, eg., by glycosylation, acetylation, pegylation, phosphorylation, amidation, derivatization by known protecting/blocking groups, proteolytic cleavage, linkage to a cellular ligand or other protein, etc. Furthermore, any of numerous chemical modifications may also be made using known techniques. These include specific chemical cleavage, acetylation, formylation, metabolic synthesis of tunicamycin, etc. Additionally, the derivative may contain one or more non-classical amino acids.

The antibodies of the present invention may be monospecific, bispecific, trispecific, or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. For example, see PCT publications WO 93/17715; WO 92/08802; WO 91/00360; WO 92/05793; Tutt et al., J. Immunol. 147: 60-69, 1991; U.S. Pat. Nos. 4,474,893; 4,714,681; 4,925,648; 5,573,920; 5,601,819; and Kostelny et al. J. Immunol. 148: 1547-1553, 1992).

In one embodiment, the antibodies of the present invention may act as agonists or antagonists of a polysaccharide transferase. In further embodiments, the antibodies of the present invention may be used, for example, to purify, detect, and target the polysaccharide transferases of the present invention, including both in vitro and in vivo diagnostic methods. For example, the antibodies have use in immunoassays for qualitatively and quantitatively measuring levels of polysaccharide transferase polypeptide in biological samples. See, e.g., Harlow et al., Antibodies: A Laboratory Manual (Cold Spring Harbor Laboratory Press, 2nd ed. 1988).

Antibodies may be generated using methods known in the art, such as in vivo immunization, in vitro immunization, and phage display methods. For example, see Bittle et al. (J. Gen. Virol. 66: 2347-2354, 1985).

If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For example, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde.

For example, polyclonal antibodies to a polysaccharide transferase polypeptide can be produced using methods known in the art. For example, animals such as rabbits, rats or mice may be immunized with either free or carrier-coupled peptides. For instance, intraperitoneal and/or intradermal injection of emulsions containing about 100 micrograms of peptide or carrier protein may be used to induce the production of sera containing polyclonal antibodies specific for the antigen. Various adjuvants may also be used to increase the immunological response, depending on the host species, for example, Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Several booster injections may be needed, for example, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods known in the art.

As another example, monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988) and Hammerling et al., in: Monoclonal Antibodies and T-Cell Hybridomas (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Antibody fragments which bind to a polysaccharide transferase of the present invention may also be generated by known techniques. For example, Fab and F(ab′)2 fragments may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

The antibodies of the present invention can also be generated using various phage display methods known in the art. Examples of phage display methods that can be used to make the antibodies of the present invention include those disclosed by Brinkman et al. (J. Immunol. Methods 182: 41-50, 1995), Ames et al. (J. Immunol. Methods 184: 177-186, 1995), Kettleborough et al. (Eur. J. Immunol. 24: 952-958, 1994), Persic et al. (Gene 187: 9-18, 1997), Burton et al. (Advances in Immunology 57: 191-280, 1994); PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

After phage selection, antibody coding regions from the phage can be isolated and used to generate whole antibodies or any other desired antigen binding fragment, and expressed in any desired host, including mammalian cells, insect cells, plant cells, yeast, and bacteria. For example, techniques to recombinantly produce Fab, Fab′ and F(ab′)2 fragments can also be employed using methods known in the art such as those disclosed in PCT publication WO 92/22324; Mullinax et al. (BioTechniques 12(6): 864-869, 1992); and Sawai et al. (AJRI 34:26-34, 1995); and Better et al. (Science 240: 1041-1043, 1988).

Examples of techniques which can be used to produce single-chain Fvs and antibodies include those described in U.S. Pat. Nos. 4,946,778 and 5,258,498; Huston et al. (Methods in Enzymology 203: 46-88, 1991); Shu et al. (Proc. Natl. Acad. Sci. USA 90: 7995-7999, 1993); and Skerra et al. (Science 240: 1038-1040, 1988).

In a seventh aspect, the present invention also contemplates an aptamer that binds to the polysaccharide transferase of the first aspect of the invention.

Nucleic acid aptamers that bind to a particular protein (such as a polysaccharide transferase) may be produced using methods known in the art. For example, in-vitro selection methods (eg. see Ellington and Szostak, Nature 346(6287): 818-22, 1990) and SELEX methods (eg. see Tuerk and Gold, Science 249(4968): 505-510, 1990) may be used. Further details relating to the production and selection of aptamers may also be found in the review of Osborne and Ellington (Chem Rev 97(2): 349-370, 1997).

The present invention is further described by the following non-limiting examples:

### BRIEF DESCRIPTION OF THE FIGURES

FIG. 1 SDS-PAGE, IEF and a primary sequence of the purified HvXET5. (A), A monodisperse HvXET5 (2.4 μg of protein) after 5 purification steps, detected with a colloidal Coomassie Brilliant Blue G-250. (B), IEF of the purified HvXET5; the activity of the enzyme was detected by zymogram detection with TXG and XGO-SR. The molecular mass standards, pH boundaries of the IEF gel, and the pI point of HvXET5 are indicated. (C), A primary sequence of HvXET5 (TrEMBL accession number P93668). The first 30 amino acid residues (underlined) were determined by Edman degradation.

FIG. 2 pH and Temperature dependences of relative activities of HvXET5. (A), pH and (B), temperature dependences were determined radiometrically after 15 min incubation at the indicated pH or temperatures values.

FIG. 3 Rate of synthesis of HEC:XGO-SR conjugate and transfer of XXXGol onto HEC:XGO-SR by HvXET5. (A), The synthesis of high molecular mass HEC:XGO-SR from unlabeled HEC and low molecular mass, fluorescent XGO-SR was monitored by HPLC for up to 24 h. Progressive formation of high molecular mass HEC:XGO-SR indicates that HEC and XGO-SR molecules are linked together by the enzymic action of HvXET5. The grey shaded area indicates the material that was pooled for the reverse reaction shown in panel B. (B), In the reverse reaction, the progressive transfer of fluorescent label from the HEC:XGO-SR conjugate (the grey shaded area in panel A) to a non-fluorescent XXXGol acceptor was followed by HPLC up to 16 h. The positions of molecular mass standards dextran (500 kDa), polyethylene glycols 8000 and 1450 and glucose (180) are indicated. (C), A schematic representation of the transfer reactions shown in panels A and B and catalysed by HvXET5. Dashes indicate covalent linkages.

FIG. 4 Characterization of HEC:XXXG-SR conjugate synthesized by HvXET5. (A), HPLC chromatogram of high molecular mass HEC:XXXG-SR synthesized from unlabeled HEC and fluorescent XXXG-SR by HvXET5; the reaction proceeded for 48 h. (B), Purification by HPLC of three major oligosaccharide fractions from the degradation products of HEC:XXXG-SR conjugate. In set, hydrolysis of HEC:XXXG-SR by T. reesei (1,4)-β-D-glucan endohydrolase. The blue shaded triangle indicates the fractions that were pooled and separated. (C) MALDI-TOF mass spectra of HEC:XXXG-SR-derived oligosaccharide peaks 1-3 from panel B show the presence of XXXG-SR (panel 1), Glc-XXXG-SR (panel 2), Glc-Glc:XXXG-SR (panel 3, black arrows) and Glc-Glc-Glc:XXXG-SR (panel 3, dark blue arrows). The non-reducing terminal glucosyl residues originated from HEC and ancillary peaks showed that some of the released oligosaccharides carried hydroxyethyl groups of m/z 45. (D), Monoisotopic m/z values for non-substituted or mono-hydroxyethylated Glc:XXXG-SR. The number 2 on the right-hand side of panel D corresponds to the number 2 in panel C. The positions of molecular mass standards specified in FIG. 3 are indicated.

FIG. 5 Characterization of (1,3;1,4)-β-D-glucan:XXXG-SR conjugate synthesized by HvXET5. (A), HPLC chromatogram of high molecular mass, fluorescent (1,3;1,4)-β-D-glucan:XXXG-SR synthesized after 144 h from unlabeled (1,3;1,4)-β-D-glucan and XXXG-SR by HvXET5. The material eluting between 8-12 min was pooled for hydrolysis by B. subtilis (1,3;1,4)-β-D-glucan endohydrolase. Fluorescence and ELSD profiles have non-linear detection responses. (B), Purification by HPLC of five major oligosaccharides released from (1,3;1,4)-β-D-glucan:XXXG-SR by (1,3;1,4)-β-D-glucan endohydrolase. (C), MALDI-TOF mass spectra of (1,3;1,4)-β-D-glucan-derived oligosaccharide conjugates 1-5 from panel B showed the presence of XXXG-SR with 2-6 covalently attached glucosyl residues at its non-reducing termini. The position of XXXG-SR is marked by an arrow. The positions of molecular mass standards of (1,3;1,4)-β-D-glucan (40 kDa), polyethylene glycol 1450 and glucose (180) are indicated.

FIG. 6 A barley xyloglucan xyloglucosyl transferase HvXET5 covalently links xyloglucan, cellulose and (1,3;1,4)-β-D-glucan. The polymers xyloglucan, cellulose and (1,3;1,4)-β-D-glucan are shown in blue, brown, and green. The distance between individual cellulosic microfibrils (d) could be altered after HvXET5 links xyloglucan and cellulosic microfibrils.

### EXAMPLE 1

### Materials Used

Ampholine™ isoelectric focusing (IEF) polyacrylamide gels (pH range 3.5-9.5), molecular mass marker proteins (20-94 kDa) and dextran 500 were from GE Healthcare Biosciences (NSW, Australia), Bio-Gel P-60, Phenyl-Sepharose and pI marker proteins 4.45-9.6 were from Bio-Rad Laboratories (Hercules, Calif., USA), Microcon microconcentrators were from Amicon (Beverly, Mass., USA), Whatman 3MM paper was from Whatman (Brentford, UK), Miracloth (22-25 μm pore size) was from Calbiochem (San Diego, Calif., USA) and ampholines were from Serva (Heidelberg, Germany). Phenylmethylsulfonyl fluoride, 2-mercaptoethanol, glucose, ethylenediamine tetraacetic (disodium salt, dihydrate) (EDTA), BSA (fraction V), polyethylene glycols 8000 and 1450, bovine serum albumin, pectin (citrus fruit), esterified pectin (K salt; citrus fruit), polygalactouronic acid, and laminarin (from Laminaria digitata) were supplied by Sigma Chemical Company (St. Louis, Mo., USA). Lissamine rhodamine B sulfonyl chloride (sulforhodamine, SR) was from Acros Organics (Morris Planes, N.J., USA), the Coomassie Protein Assay Reagent was from Pierce (Rockford, Ill., USA), EcoLume scintillation fluid was from MP Biomedicals (Irvine, Calif., USA), chromatography 3MM paper was from Whatman (Brentford, Middlesex, UK) and acetonitrile was from BDH Laboratory Supplies (Poole, England). Barley (1,3;1,4)-β-D-glucans (average molecular masses of 450 and 40 kDa), β-D-galactans (from lupin and potato), lichenin, ivory nut mannan, konjac glucomannan, barley arabinoxylan, tamarind xyloglucan (TXG), rhamnogalactouronan (soybean pectic fibre), xyloglucan-derived heptasaccharide (XXXG), its reduced form XXXGol, and (1,3;1,4)-β-D-glucanase from Bacillius subtilis were from Megazyme (Bray, Ireland). Carboxymethyl cellulose (CMC) of degree of substitution 0.54 was from Imperial Chemical Industries (Dingley, Australia), arabinogalactan protein (from gum arabic) was from Aldrich Chemical Corporation (Milwaukee, Wis., USA), and cello-oligosaccharides (CEO) of degree of polymerization (DP) of DP 2-6 and laminari-oligosaccharides (LAO) of DP 2-6, were from Seikagaku Corporation (Tokyo, Japan). Cello-heptaose and cello-octaose were prepared by acid hydrolysis from Antigum CS6 (System Bio-Industries, Paris, France). Hydroxyethylcellulose (HEC) of medium viscosity (˜1500 mPa·s, 5% in H₂O at 20° C.), of average molecular mass 450 kDa and of a degree of substitution approximately 0.3, was from Fluka Biochemica (Buchs, Switzerland). Beechwood 4-O-methyl-(1,4)-β-D-glucuronoxylan was from Institute of Chemistry (Slovak Republic), a low viscosity locust-bean gum galactomannan was donated by Dr Peter Biely (Institute of Chemistry), sulfuric acid swollen cellulose of average molecular mass of 12-15 kDa and a degree of substitution approximately 0.25, was provided by Professor Bruce Stone (La Trobe University, Australia), and 1,4-p-D-glucan endohydrolase EGII from Trichoderma reesei was kindly donated by the late Dr Marianne Hayn (University of Graz, Austria).

### EXAMPLE 2

### Methods—Extraction of HvXET5

Barley (Hordeum vulgare L., cv. Clipper) (2 kg dry weight) was surface sterilised for 10 min in 0.1% (w/v) NaOCl, washed successively with tap water, 0.5 M NaCl and sterile water, and steeped for 24 h in sterile water containing chloramphenicol (100 μg/ml), neomycin (100 μg/ml), penicillin G (100 U/ml) and nystatin (100 U/ml). Germinating grains were maintained at approximately 40% (w/w) moisture content by regular application of fresh antibiotic solution for 7 days at 21±2° C. in the dark. Bacterial or fungal contamination of the grains was not evident at any stage during this period. The germinated grain and young seedlings were homogenised at 4° C. in 2.0 volumes of homogenization buffer, pH 6, containing 0.1 M imidazole-HCl buffer, 1M NaCl, 2 mM EDTA, 1 mM 2-mercaptoethanol and 1 mM phenylmethylsulfonyl fluoride (buffer A), in a Waring Blender for 6×1 min intervals with intermittent cooling (2 min) on ice. The homogenate was held for 1 h at 4° C. to extract proteins, insoluble material was removed by centrifugation (4000 g, 60 min, 4° C.), and the extract filtered through Miracloth. The extract was precipitated to 90% with solid (NH₄)₂SO₄, the precipitate collected (8000 g, 45 min, 4° C.) and resuspended in 4 litres of buffer A (without NaCl). The extract was stored in 0.5 litre aliquots at −20° C.

### EXAMPLE 3

### Methods—Purification of HvXET5

The HvXET5 enzyme was purified from extracts of seven-day-old barley seedlings using Sepharose Q Phenyl-Sepharose, chromatofocussing on PBE-94 and size-exclusion chromatography on Bio-Gel P-60, as shown in Table 2.

TABLE 2
Enzyme yields and purification factors of HvXET5 from 7-day-old seedlings

| | Yield | | Specific | | Purification |
|---|---|---|---|---|---|
| | Protein Activity ^{a} | | Activity | Recovery ^{b} | factor ^{c} |
| Purification step | mg | Pkat | pkat · mg⁻¹ | % | -fold |
| Crude homogenate | 6,597 | 7,777 | 1.2 | 100 | 1.0 |
| (1M NaCl extract) | | | | | |
| 90% (NH₄)₂SO₄ | 5,375 | 8,512 | 1.6 | 110 | 1.3 |
| Sepharose Q, pH 6.8 | 1,254 | 7,771 | 6 | 100 | 5 |
| Phenyl-Sepharose, pH 6.0 | 450 | 6,909 | 15 | 89 | 13 |
| PBE-94, pH 5.0-8.3 | 50 | 2,996 | 60 | 39 | 50 |
| Bio-Gel P-60, pH 7.0 | 0.1 | 363 | 3,630 | 5 | 3,025 |

^{a} As a total enzyme activity in selectively pooled fractions assayed radiometrically.
^{b} Recoveries are expressed as % of a total enzyme activity in a crude homogenate.
^{c} Purification factors are based on specific activities.

The activity of HvXET5 during enzyme purification was determined radiometrically at 30° C. in 100 mM succinate or ammonium acetate buffers, pH 6.0, containing 5 mM calcium chloride, 0.3% (w/v) TXG and [³H]-labelled xyloglucan-derived saccharide heptaitol (XXXGol, specific radioactivity 83 MBq·μmol⁻¹) (Fry, et al., Biochem. J. 282, 821-828, 1992) with approximately 30,000 dpm per reaction mixture. The radioactivity incorporated in reaction products was counted on 2×1 cm Whatman chromatography 3MM paper strips in plastic vials and using LSC6500 Scintillation counter (Beckman, Fullerton, USA) with approximately 48% efficiency for tritium, using EcoLume scintillation fluid, and with 70% quenching. Enzyme activity of HvXET5 during purification is expressed in katals, where 1 kat represents 1 mol of product formed per s; specific activity is expressed in pkat·mg⁻¹ protein.

### EXAMPLE 4

### Methods—Protein Determination, SDS-PAGE and Amino Acid Sequencing

Protein concentration determinations during purification and characterization of HvXET5 and SDS-PAGE, were performed as described by Hrmova et al. (Biochem. J. 399, 77-90, 2006). During HvXET5 purification, protein was detected with colloidal Coomassie Brilliant Blue G-250 in methanol for 30 h at ambient temperature (Neuhoff et al., Electrophoresis 9, 225-262, 1988). This staining technique detects approximately 6-10 ng protein per band or 0.7 ng per mm². Automated amino acid sequence analysis of HvXET5 was performed by Edman degradation as described by Hrmova et al. (J. Biol. Chem. 271, 5277-5286, 1996). A single primary sequence was detected, with no secondary sequence. Clear signals for each phenylthiohydantoin amino acid residue derivative were detected.

### EXAMPLE 5

### Methods—Isoelectric Focusing

The crude protein extracts and purified preparations were separated on a flatbed IEF apparatus (GE Healthcare Biosciences) in 1 mm polyacrylamide gels using a pH gradient of 3.5-9.5. Pre-focused gels were run at 600 V for 30 min, followed by 800V for a further 20 min. Proteins were detected with a Coomassie Brilliant Blue dye after the gels were fixed in 20% (w/v) trichloroacetic acid. Apparent pI values were estimated by reference to marker proteins with pI values of 4.45-9.6. Enzyme activity in gels was detected by overlaying the separation gels with a 1.5-mm 1.3% (w/v) agarose detection gel containing 0.2% (w/v) TXG and 5-10 μM of SR-labeled xyloglucan-derived oligosaccharides XGO-SR (XXXG-SR, XXLG-SR, XLLG-SR molar ratios were 1:1.6:1.8) (Fry, Plant J. 11, 1141-1150, 1997; Farkas et al. Plant Physiol. Biochem. 43, 431-435, 2005) in 0.1 M succinate buffer, pH 6, containing 5 mM calcium chloride. The separation gels contacted with detection gels were incubated for 1-5 h at 30° C., depending on the activity of the preparation under investigation. The detection gels were immediately fixed and de-stained in 60% (v/v) ethanol containing 5% (v/v) formic acid. The detection gel with fluorescent zones was evaluated under a UV lamp at 366 nm.

### EXAMPLE 6

### Methods—pH Optimum and Enzyme Stability

The effect of pH on the activity of HvXET5 was determined by incubating 1 nM HvXET5 at 30° C. for 60 min in 50 mM citric acid/100 mM sodium dihydrophosphate (McIlvaine) buffers, pH 4.0-8.5 in the presence of 0.02% (w/v) BSA. A comparison of succinate, ammonium acetate or sodium phosphate buffers, each at 50-200 mM, indicated that HvXET5 activity was unaffected by the ionic strength of these buffers. The thermal stability of HvXET5 was determined after 15 min incubation at 0-70° C. The freeze/thaw stability of HvXET5 at 1 nM concentration was determined after three cycles of freezing (−80° C.) and thawing (4° C.), each of 3 min duration. Activity was subsequently measured at 30° C. in 100 mM ammonium acetate buffer, pH 6, containing 5 mM calcium chloride, without and with the addition of 10% (v/v) glycerol. Enzyme activity was determined radiometrically as specified above, and expressed as % activity relative to maximal activity. Assays were performed in triplicate and standard errors of 8-14% were observed.

### EXAMPLE 7

### Methods—Effects of Divalent Cations

The effect of Ca²⁺ (as calcium chloride) and Mg²⁺ (as magnesium sulfate), both in 0-15 mM concentration ranges, and of EDTA in a 0-20 mM concentration range, were determined by incubating 1 nM HvXET5 in 100 mM succinate buffer, pH 6. Enzyme activities were determined radiometrically as specified above, in triplicate and with a standard error of 8-10%.

### EXAMPLE 8

### Methods—Substrate Specificities

The incubation mixtures contained 1.2% (w/v) soluble polysaccharides as donor substrates, and as acceptor substrates either 23-27 μM XGO-SR, SR-labelled cello-oligosaccharides (CEO-SR) (DP 2-8) or SR-labelled laminari-oligosaccharides (LAO-SR) (DP 2-6) (Farkas et al. Plant Physiol. Biochem. 43, 431-435, 2005) in 100 mM ammonium acetate buffer, pH 6, containing 5 mM calcium chloride and 0.5-1 nM purified HvXET5. The molar ratios of individual oligosaccharides in the two oligosaccharide-SR mixtures were 1:0.78:0.62:0.41:0.16:0.03:0.013 for C2-SR to C8-SR, and 1.0:1.7:0.87:0.41:0.2 for L2-SR to L6-SR. All incubations proceeded for 18 h at 30° C. Enzymes inactivated by boiling for 3 min served as controls. The efficiency of transfer of selected polysaccharides onto fluorescent acceptors was determined by size exclusion HPLC. The SR-labelled oligosaccharides and polysaccharides were detected following HPLC by fluorescence detection (excitation 568 nm and emission 584 nm) or by evaporative light scattering detection (ELSD) at 568 nm, and by MALDI TOF mass spectrometry analyses. Enzyme activities were determined by integrating peak areas, after subtracting background level obtained from boiled enzyme control reactions. Relative activities of HvXET5 are expressed as % of activity observed with TXG as a donor substrate and XGO-SR as an acceptor. In all instances assays were performed in duplicate with standard errors of 8-12%. Detection limits of the fluorescence assays and ELSD were better than 0.1 μmol XGO-SR or CEO-SR, or 1·10⁻⁵% of the amount of XGO-SR or CEO-SR acceptors used in standard enzyme reactions, with a standard error of 6%. The efficiency of transfer of selected polysaccharides onto [³H]-XXXGol was further evaluated by ascending chromatography in 60% (v/v) ethanol on Whatman chromatography 3MM paper strips, and radioactivity in paper strips was determined by liquid scintillation counting as specified above.

### EXAMPLE 9

### Methods—Preparation of HEC:XXXG-SR and (1,3;1,4)-β-D-Glucan:XXXG-SR Conjugates

XXXG-SR was prepared as described by Fry (Plant J. 11, 1141-1150, 1997) and Farkas et al. (Plant Physiol. Biochem. 43, 431-435, 2005) and purified on a reversed phase column with a water/acetonitrile gradient. A relatively homogenous HEC and (1,3;1,4)-β-D-glucan fractions (collected after separations by size-exclusion chromatography) at 0.8% (w/v) concentrations were dissolved in 100 mM ammonium acetate buffer, pH 6, containing 5 mM calcium chloride, and incubated with 8 μM XXXG-SR, in the presence of 4 nM purified HvXET5 at 30° C. with shaking. Every 16 h (HEC) or 24 h [(1,3;1,4)-β-D-glucan] a fresh HvXET5 enzyme (¼ of the original amount) was added. The reactions were terminated by boiling after 48 h and 144 h, for HEC and (1,3;1,4)-β-D-glucan, respectively.

The rates of synthesis of HEC:XGO-SR conjugate by HvXET5 at 1 nM concentration were followed for 0, 16 and 24 h. The rates of transfer of 130 μM XXXGol onto 0.1% (w/v) HEC:XGO-SR conjugate by HvXET5 (1 nM) were followed for 0, 6, and 16 h. Boiled enzymes served as controls in all instances. The products were analysed by HPLC with fluorescent and evaporative light scattering (ELSD) detectors.

### EXAMPLE 10

### Methods—Characterization of HEC:XXXG-SR and (1,3;1,4)-β-D-Glucan:XXXG-SR Conjugates

Approximately 0.5 mg HEC:XXXG-SR or (1,3;1,4)-β-D-glucan:XXXG-SR was dissolved in 50 mM ammonium acetate buffer, pH 5 and incubated with 10 nM of the purified 1,4-p-D-glucan endohydrolase from Trichoderma reesei (family GH5 glycoside hydrolase; Suurnäkki, et al., Cellulose 7, 189-209, 2000) or 1 nM of the purified (on Bio-Gel P-60) (1,3;1,4)-β-D-glucanase (a family GH16 glycoside hydrolase; Hahn et al., Eur. J. Biochem. 232, 849-858, 1995) from Bacillus subtilis for 1 h at 30° C. The reactions were stopped by boiling and the hydrolysis products of HEC:XXXG-SR and (1,3;1,4)-β-D-glucan:XXXG-SR were separated by HPLC and analysed by MALDI-TOF mass spectrometry.

### EXAMPLE 11

### Methods—HPLC Analysis

Native polysaccharides were fractionated by size-exclusion chromatography on either a P3000 or P4000 PolySep GFC columns (particle size not specified, 300×7.8 mm) (Phenomenex, Torrance, Calif., USA) with water or 100 mM ammonium acetate as eluant at a flow rate of 0.8 ml/min. Fractionations of the mixture of XGO-SR (XXXG-SR, XXLG-SR and XLLG-SR) and the hydrolysis products of HEC:XXXG-SR and (1,3;1,4)-β-D-glucan:XXXG-SR were performed on a Hypersil ODS column (5 μm, 250×2.1 mm) (Thermo Electron Corporation, Waltham, Mass., USA) with a linear gradient of 23.45-26.65% aqueous acetonitrile at a flow rate of 0.2 ml/min. A model 1090 liquid chromatograph with diode-array detector, controlled by ChemStation software (Agilent Technologies, Palo Alto, Calif., USA), and fluorescence (model RF-10AXL, Shimadzu, Kyoto, Japan) and ELSD (model 800, Alltech Associates Inc., Deerfield, Ill., USA) connected in series to the 1090 DAD, were used for analyses of enzymatic reactions. The eluant flow from the fluorescence detector to the ELSD was split in the ratio 5 (to collect) to 1 (ELSD). The ELSD was operated at 40° C. and a nitrogen pressure of 1.5 bar and the column temperature was 21° C. Size exclusion HPLC of SR-labelled polysaccharides and oligosaccharides were carried out on a BioSep SEC S3000 column (5 μm, 300×7.8 mm); the eluant was 100 mM ammonium acetate in 20% (v/v) acetonitrile at a flow rate of 1.0 ml/min.

### EXAMPLE 12

### Methods—MALDI-TOF Mass Spectrometry Analyses

MALDI TOF spectra were acquired using a Bruker ultraflex II MALDI TOF/TOF mass spectrometer (Bruker Daltonik GmbH, Bremen, Germany) operating in a reflectron mode.

Samples (1 μl) dissolved in water were mixed with 1 μl of a 5 g/l solution of dihydroxybenzoic acid in 1% (v/v) phosphoric acid, and spotted on a matt-steel target plate. External calibration was performed using peptide standards (Bruker Daltonik GmbH, Bremen, Germany), which were analysed under the same conditions. Spectra were acquired using between 800-3,000 laser shots. The ionization voltages were IS1=25.0 kV, IS2=21.7 kV and lens=8.2 kV. The mass spectrometer was calibrated with XXXG-SR, XXLG-SR and XLLG-SR that were purified by tandem normal phase and size-exclusion HPLC chromatography.

### EXAMPLE 13

### Results—Purification of the Barley HvXET5

The barley HvXET5 enzyme was purified approximately 3,000-fold from extracts of 7-day-old barley seedlings, using ammonium sulphate precipitation, ion exchange and hydrophobic chromatography, chromatofocussing and size exclusion chromatography (see Table 1). The numbering of the barley XET isoenzymes is based upon the gene nomenclature proposed by Strohmeier et al. (Protein Sci. 13, 3200-3213, 2004). The specific activity of the purified HvXET5 was 3,630 pkat·mg⁻1.

It was necessary to use IM NaCl in the imidazole buffer for efficient enzyme extraction, presumably because the XET enzymes are tightly associated with cell wall material. The key steps during the purification of HvXET5 were chromatofocussing on PBE-94 and chromatography on Bio-Gel P-60, where several HvXET isoenzymes were separated from each other and from major contaminating proteins (see Table 1). The enzymes bound strongly to Phenyl-Sepharose and were not eluted by a 3-0 M linear gradient of NaCl. A 30-70% linear gradient of ethylene glycol was required for elution, suggesting that the enzymes were highly hydrophobic. The HvXET5 isoenzyme also bound to Bio-Gel P-60 and 0.01% (v/v) Tween 20 and 0.2 M NaCl were required to elute the enzyme from the column.

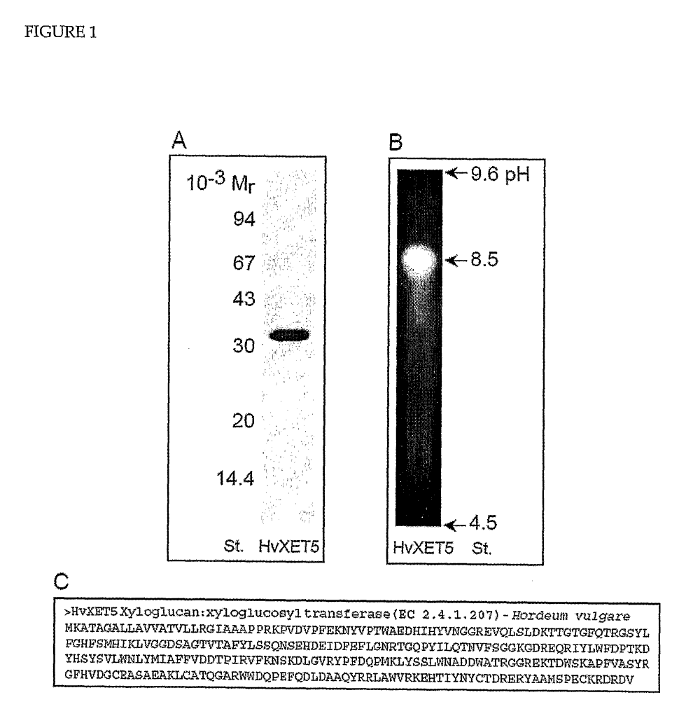

The final purified enzyme preparation showed a single band of 34 kDa on SDS gels at high protein loadings (FIG. 1A), with no other protein species present. These data indicated that contaminating proteins accounted for less than 6 ng, or 0.25% of the protein used for SDS-PAGE analysis. Furthermore, a single protein and activity band of pI 7.6 was detected on an isoelectric focusing gel, and on a zymogram detection gel containing TXG and SR-labelled xyloglucan oligosaccharides XGO-SR (FIG. 1B). A single sequence (SEQ ID NO: 1) was detected during NH2-terminal amino acid sequence analysis of the purified HvXET5 enzyme, where a 97% yield of a phenylthiohydantoin-alanine in the 1^{st} cycle, normalised per mole of the total protein of HvXET5, was obtained.

Another isoenzyme, HvXET6 (SEQ ID NO: 2), was partially purified from 7-day-old barley seedlings.

### EXAMPLE 14

### Results—pH Optimum and Enzyme Stability

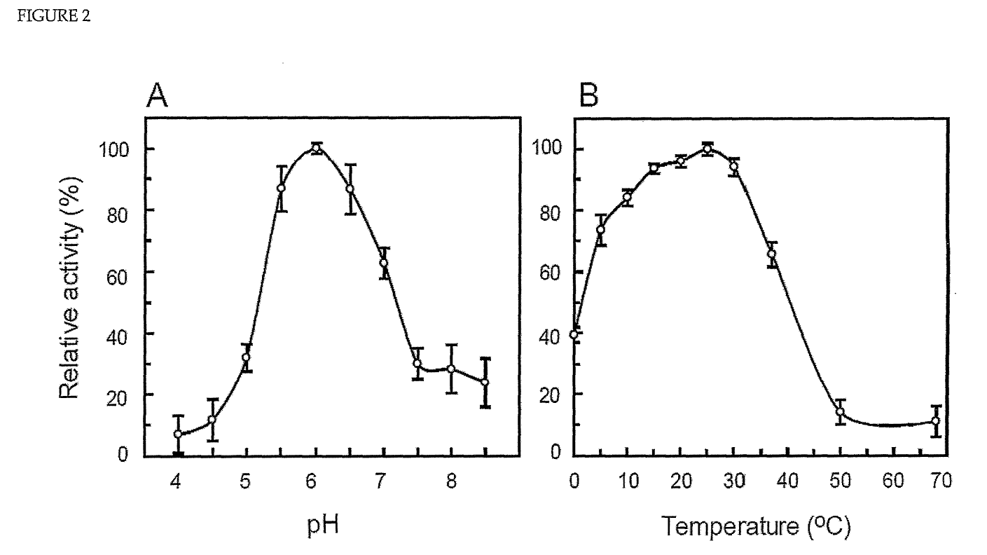

The pH optimum of the purified HvXET5 was 6.0 and the temperature optimum varied between 28° C. and 30° C. (FIG. 2). The enzyme also operated efficiently at 0° C., where it showed approximately 40% of the activity observed at 30° C.

As for the freeze/thaw stability of HvXET5, the purified enzyme retained its activity for at least a year when stored at −20° C., and did not lose activity after several freeze-thaw cycles. The addition of 10% (v/v) glycerol had no affect on HvXET activity after several freeze-thawing cycles.

### EXAMPLE 15

### Results—The Effects of Divalent Cations

Effects of the divalent cations Ca²⁺ and Mg²⁺ were tested on the activity of HvXET5 under optimal conditions. While Ca²⁺ at concentrations between 5 and 15 mM stimulated HvXET5 activity by approximately 7-8%, Mg²⁺ inhibited the activity of HvXET5 by 3-4% in the same concentration range. The chelating agent EDTA inhibited the activity of HvXET5 by approximately 30% in 2-20 mM concentration ranges.

### EXAMPLE 16

### Results—Substrate Specificity of the Purified HvXET5

Xyloglucan oligosaccharides (XGO-SR) and cello-oligosaccharides (CEO-SR) fluorescently labelled with sulforhodamine (SR) were used as acceptor substrates for the purified HvXET5. Transferase activity was observed when tamarind xyloglucan (TXG), hydroxyethylcellulose (HEC), sulfuric acid-swollen cellulose and barley (1,3;1,4)-β-D-glucan were used as donor polysaccharides, as shown in Table 3.

TABLE 3
Donor and acceptor substrate specificities of HvXET5

| | Acceptor | | Relative |
|---|---|---|---|
| Donor substrate | substrate | Activity^{a} | activity^{b,c} (%) |
| TXG | XGO-SR | 12,100 | 100 |
| | CEO-SR | 13 | 0.1 |
| HEC | XGO-SR | 5,347 | 44.2 |
| | CEO-SR | 30 | 0.2 |
| Sulfuric acid swollen cellulose | XGO-SR | 605 | 5.0 |
| | CEO-SR | nd^{d} | nd^{d} |
| Carboxymethyl cellulose | XGO-SR | 50 | 0.4 |
| | CEO-SR | nd^{d} | nd^{d} |
| Barley 1,3;1,4-β-D-glucan | XGO-SR | 9 | 0.2 |
| | CEO-SR | 10 | 0.1 |
| Locust bean gum galactomannan | XGO-SR | 6 | 0.1 |
| | CEO-SR | nd^{d} | nd^{d} |

^{a}Enzyme activities (expressed as Δpeak area · h⁻¹) were determined from HPLC chromatograms.
^{b}Relative activities are expressed as % of the integrated peak area of the reaction with TXG and XGO-SR.
^{c}No transferase activities were detected with laminarin, lichenin, pustulan, barley arabinoxylan, Konjac glucomannan, citrus fruit pectin and esterified pectin (K salt), orange polygalactouronic acid (Na salt), soy bean pectic fibre rhamnogalactouronan, β-D-galactans (from lupin and potato) and arabinogalactan protein (from gum arabic) with XGO-SR and CEO-SR. No transferase activity was detected with any of the listed donors and LAO-SR.
^{d}Not detected.

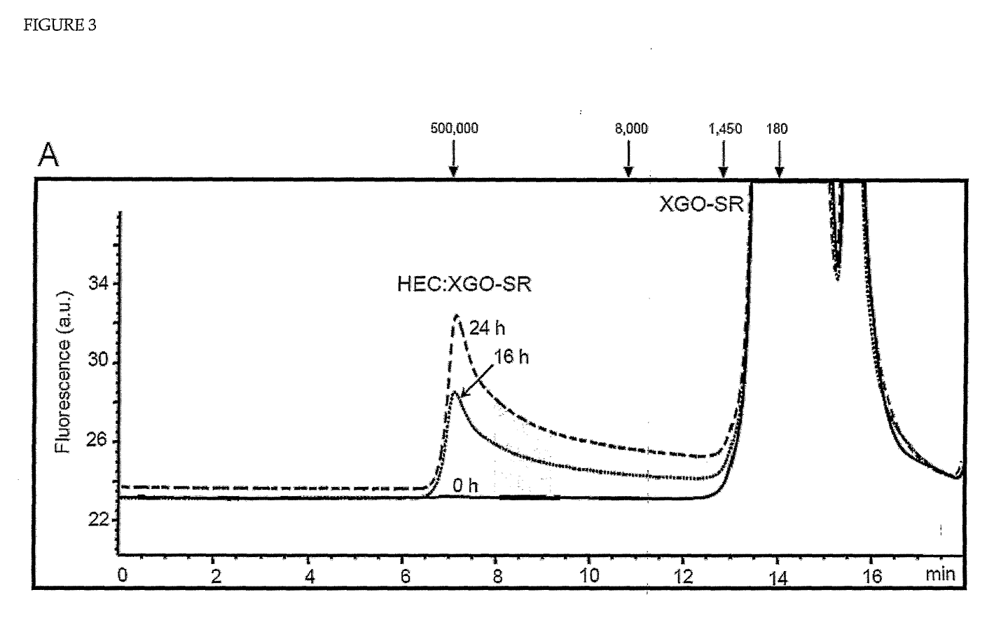

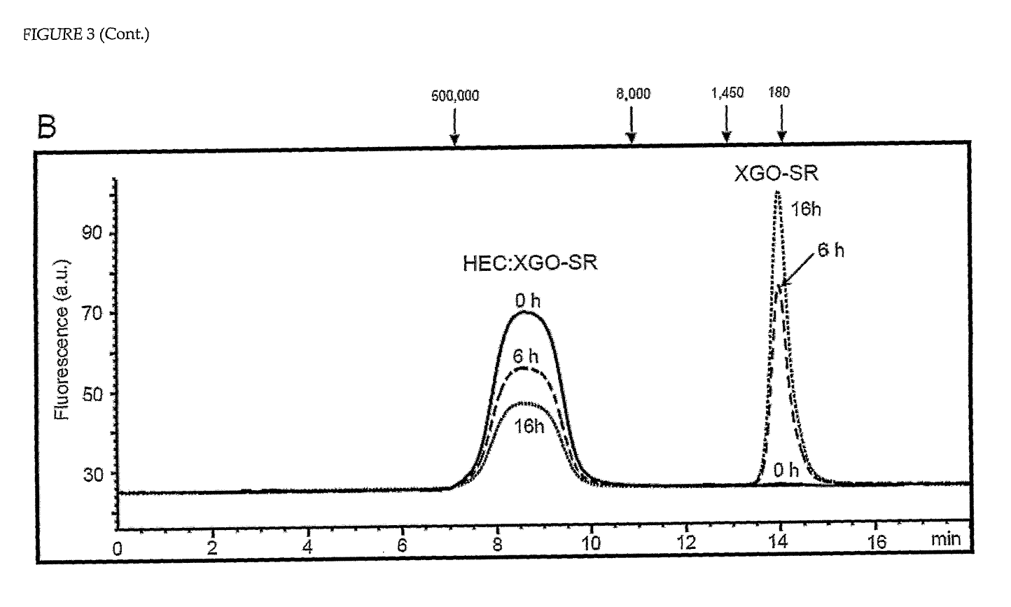

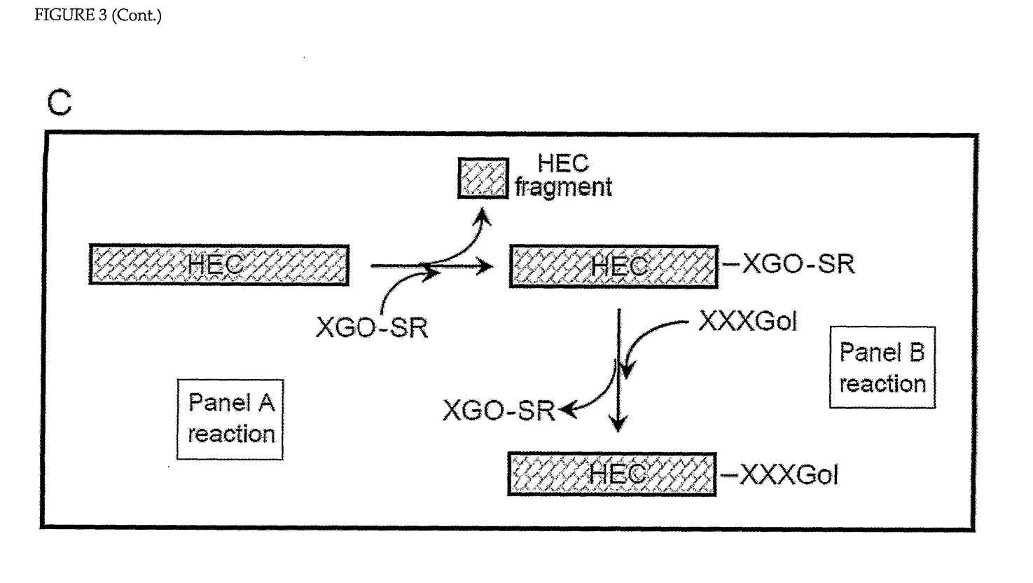

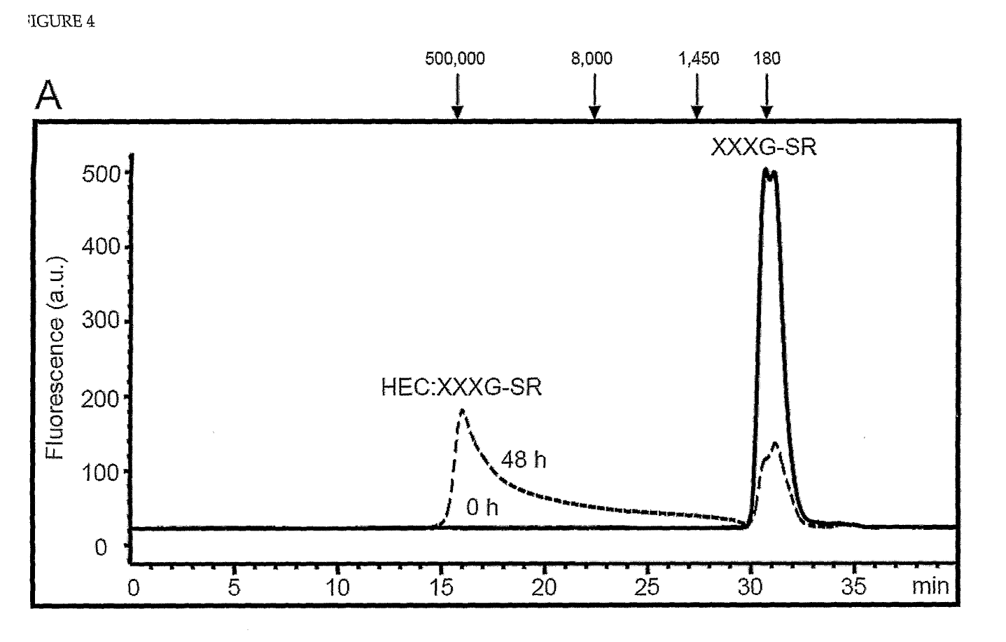

No hydrolytic activity was detected with any of the donor substrates. The transfer of non-fluorescent donor polysaccharides onto fluorescent acceptors was determined by size exclusion HPLC, where dramatic increases in molecular size of fluorescent material showed that the transfer reaction had occurred. In FIG. 3A the progressive formation over 24 h of high molecular mass, fluorescent HEC from unlabeled HEC donor substrate and the fluorescent XGO-SR acceptor molecule can be seen. In FIG. 3B, the transfer reaction by HvXET5 is shown with the high molecular mass product of the reaction presented in FIG. 3A, whereby the fluorescent component XGO-SR of the high molecular mass HEC:XGO-SR material from FIG. 3A (shaded fractions) was removed from the reducing end of the polysaccharide and replaced with the non-fluorescent oligosaccharide XXXGol. As this occurred, low molecular mass fluorescent oligosaccharides XGO-SR were progressively released. A schematic representation of the transfer reaction shown in FIGS. 3A and 3B is summarised in FIG. 3C.

The HvXET5 enzyme also catalyzes the transfer of TXG, celluloses and (1,3;1,4)-β-D-glucan onto fluorescently labelled CEO-SR, albeit at low levels (see Table 2). The fluorescence assay technique used in this study was sufficiently sensitive to confidently measure activities that were better than 0.1 μmol XGO-SR or 1.10-5% of the amount of XGO-SR acceptor used in standard enzyme reactions.

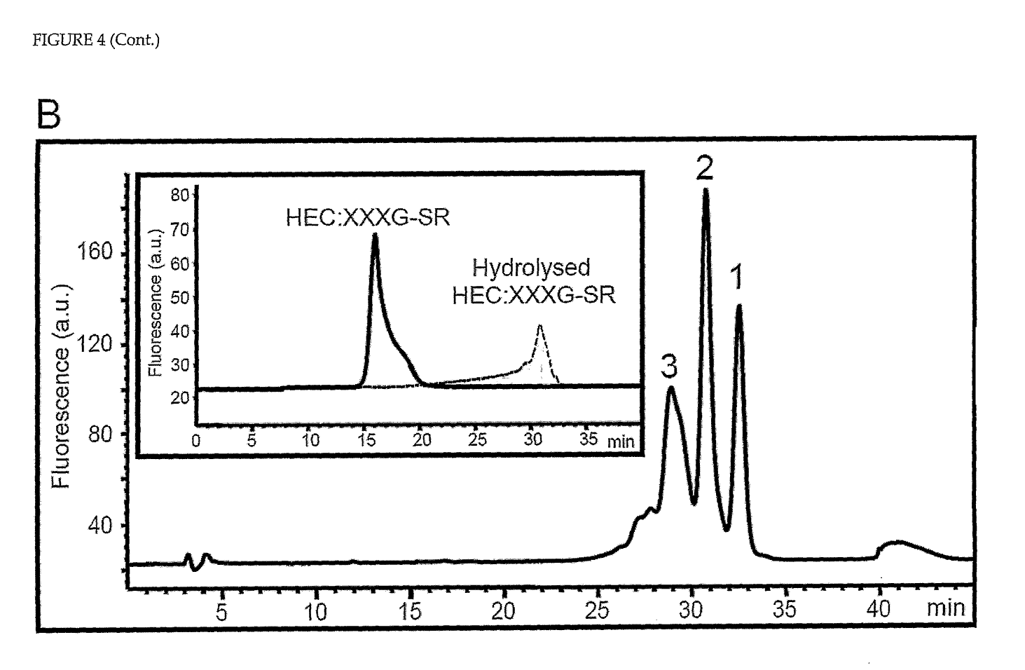

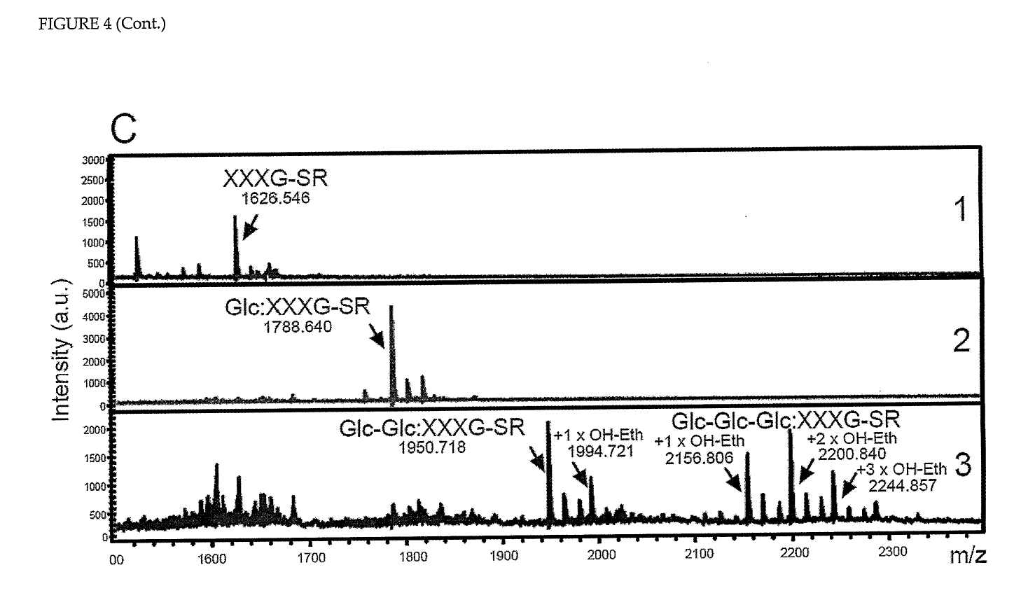

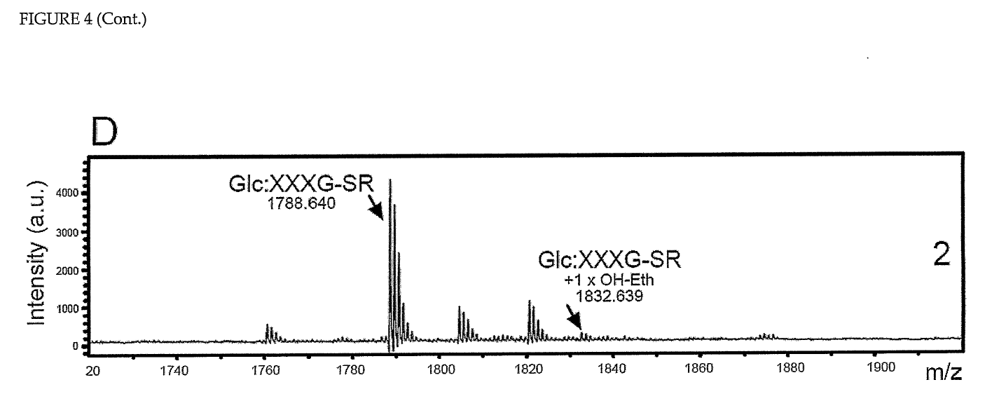

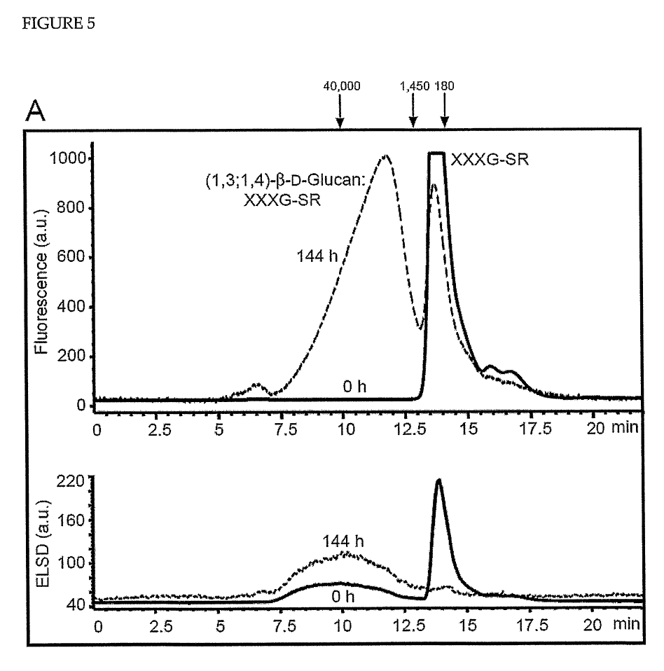

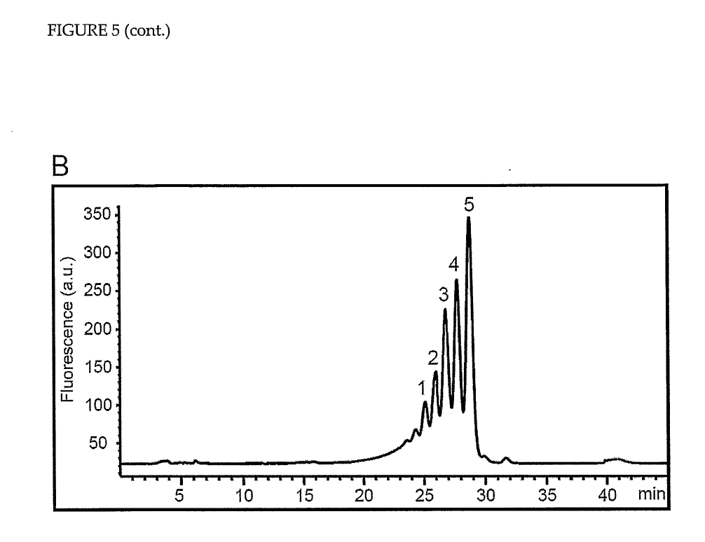

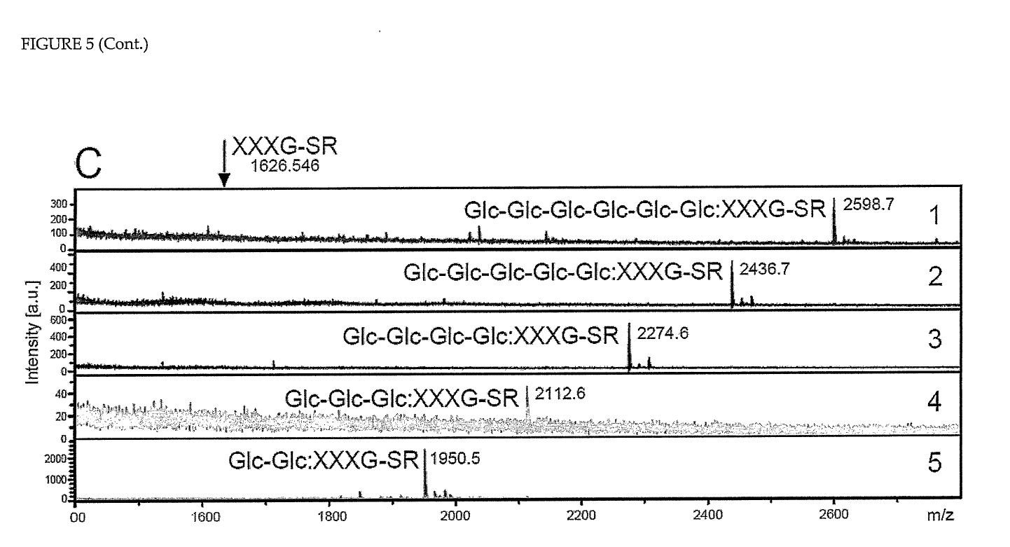

The capacity of HvXET5 to form covalent linkages between xyloglucan fragments and either cellulose or (1,3;1,4)-β-D-glucan was first shown with the oligosaccharide mixture XGO-SR and later confirmed with the single xyloglucan-derived heptasaccharide XXXG-SR (FIGS. 4-5). Similarly, formation of covalent linkages by HvXET5 between xyloglucan fragments and cellulose or (1,3;1,4)-β-D-glucan was confirmed independently using radiometric analysis with [³H]-labelled xyloglucan-derived heptaitol and paper-chromatography. The slower transfer of the (1,3;1,4)-β-D-glucan donor onto the XXXG-SR acceptor substrate, compared with donors with (1,4)-β-D-glucan backbones, probably results from the molecular kinks introduced into this substrate by the (1,3)-p-linkages (27), and hence from less favourable binding to the enzyme's active site.

In control experiments, either the acceptor or donor substrates were omitted, or the enzyme was inactivated by boiling. In no instance was any change in molecular size of fluorescent material observed. The absence of transferase activity, when either the donor or acceptor substrate was omitted, was particularly important, because it indicated that transglycosylation reactions that are often observed when polysaccharide hydrolases are incubated with high substrate concentrations, were not occurring. All experiments were performed at micromolar donor and acceptor concentrations, again to rule out the potential for transglycosylation reactions attributable to high substrate concentrations. However, it should be noted that the efficiency of such reactions would also depend upon overall substrate affinities and catalytic properties of the enzyme, together with substrate properties such as the reactivities of leaving groups.

No transferase activity was detected with laminarin, lichenin, pustulan, barley arabinoxylan, glucomannan, citrus fruit pectin or esterified pectin (K salt), orange polygalacturonic acid (Na salt), soy bean pectic fibre rhamnogalactouronan, lupin or potato β-D-galactans, or gum arabic. No transferase activity was detected with any of the potential donors and LAO-SR, which indicated that (1,3)-β-D-glucans do not bind at the acceptor site.

### EXAMPLE 17

### Results—Analysis of the Products of HvXET5 Action

To confirm the results obtained from the fluorescence assays, the high molecular mass HEC:XXXG-SR conjugate generated by incubation of the HvXET5 with XXXG-SR and non-fluorescent HEC (FIG. 4A) was partially hydrolysed with a highly purified (1,4)-β-D-glucan endohydrolase from Trichoderma reesei that contained no β-D-glucosidase or other contaminating activities. Three major fluorescent oligosaccharide fractions were released from the HEC:XXXG-SR (FIGS. 4A and 4B) during the partial hydrolysis with the (1,4)-β-D-glucan endohydrolase. MALDI-TOF mass spectrometric analyses showed that these oligosaccharides had molecular masses corresponding to XXXG-SR with one, two or three additional glucosyl residues attached (FIG. 4B). Furthermore, ancillary m/z peaks, which corresponded to Glc:XXXG-SR, Glc-Glc:XXXG-SR and Glc-Glc-Glc:XXXG-SR containing hydroxyethyl substituents (m/z 45) from the donor substrate, were also detected in the spectra (FIG. 4C and 4D) and confirmed that the parent material consisted of HEC covalently linked with the XXXG-SR.

Similarly, a (1,3;1,4)-β-D-glucan endohydrolase from Bacillus subtilis was used for digestion of the (1,3;1,4)-β-D-glucan:XXXG-SR conjugate (FIGS. 5A and 5B). The enzyme was purified from the commercially available preparation by size-exclusion chromatography on Bio-Gel P-60, to remove contaminating β-D-glucosidases (data not shown). Partial hydrolysis of the (1,3;1,4)-β-D-glucan:XXXG-SR conjugate (FIG. 5A) released a series of fluorescent oligosaccharides (FIG. 5B) that were shown by MALDI-TOF mass spectrometry to consist of the XXXG-SR acceptor substrate with 2-6 covalently attached glucosyl residues (FIG. 5C). These data indicated that the high molecular mass fluorescent material generated by the HvXET5 contained polymeric barley (1,3;1,4)-β-D-glucan covalently linked to the XXXG-SR acceptor substrate.

### EXAMPLE 18

### Discussion

Xyloglucans consist of a backbone of (1,4)-β-D-glucan substituted with xylosyl, galactosyl and fucosyl residues. The molecular sizes of xyloglucans can be altered after their deposition into the cell wall and this process is likely to be mediated by a class of enzymes broadly known as xyloglucan endotransglycosylases/hydrolases (XTHs). However, enzymes within this group can have xyloglucan endotransglycosylase (XET) activity or both xyloglucan endotransglycosylase and xyloglucan endohydrolase (XEH) activities. The XETs are abundant in the apoplastic space, they cleave the (1,4)-β-D-glucan backbone of xyloglucans and, in the case of xyloglucan endotransglycosylases (XETs), transfer the non-reducing fragment of the original substrate that remains bound to the enzyme directly onto the non-reducing terminus of another xyloglucan chain. The xyloglucan molecule that is cleaved by the enzyme initially is referred to as the donor substrate, while the xyloglucan chain to which the product of hydrolysis is transferred is known as the acceptor substrate. The transglycosylation activity of XETs can theoretically result in the disproportionation of xyloglucan molecules, such that some will increase in molecular mass while others will decrease in molecular mass.

Sequences encoding XTHs are surprisingly abundant in barley EST databases, given the relatively low levels of xyloglucans in walls of most barley tissues. There are at least 22 XTH genes in barley, about 30 in rice, about 40 in Populus trichocarpa and about 33 in Arabidopsis. In an attempt to reconcile the relatively low abundance of xyloglucans in cell walls of barley against the large number of XTH genes and their high expression levels in many tissues of barley, it was contemplated that some of the XTHs might be active on the more abundant matrix phase polysaccharides of cell walls in barley, namely the arabinoxylans and the (1,3;1,4)-β-D-glucans. A role for XTHs in the modification of highly abundant (1,3;1,4)-β-D-glucans and arabinoxylans in walls of the commelinoid monocots would be consistent with the abrupt increase in molecular size of heteroxylans that has been observed in suspension-cultured maize cells following the deposition of the polysaccharide into the walls.

Thus, molecular modeling established a potential structural connection between XTHs and (1,3;1,4)-β-D-glucan endohydrolases, and with certain (1,4)-β-D-xylan endohydrolases. The models were subsequently supported by the published 3D structure of the Populus tremula x tremuloides XET. The plant XTHs and microbial (1,3;1,4)-β-D-glucan endohydrolases are all classified in the family GH16 group of glycoside hydrolases, although a small number of microbial XEHs are also classified within families GH5, GH12, GH44 and GH74.

In an attempt to test suggestions that some barley XET enzymes could catalyze transfer of xyloglucan onto acceptors other than xyloglucans, an XET isoenzyme was purified from extracts of barley seedlings to a substantially monodisperse form. The difficulties encountered during the purification procedure of HvXET5 were largely attributable to the presence of numerous hydrophobic patches on the surface of the enzyme, as predicted from the 3D structure of the Populus XET. However, after the HvXET5 enzyme was purified, its activity remained more or less constant for at least a year at −20° C. The difficulties with enzyme purification might also explain why so few XTHs have been purified from plant tissue extracts.

The HvXET5 isoenzyme that was purified in the present work catalysed the formation of covalent linkages between celluloses such as chemically modified or paracrystalline HEC and sulfuric acid swollen cellulose, or (1,3;1,4)-β-D-glucans and xyloglucans (FIGS. 3-6). The polysaccharides are linked from reducing to non-reducing ends of donor and acceptor substrates, respectively, rather than by cross-linking of the type observed between arabinoxylan chains through esterified hydroxycinnamic acids or between pectic polysaccharides through borate. The HvXET5 activity represents a non-Leloir type of biosynthetic reaction, insofar as the energy required for the formation of the new glycosidic linkage is provided from an existing glycosidic linkage rather than from a sugar nucleotide activated donor. The data shown in FIG. 3A is particularly important with respect to the action pattern of the barley XET. The presence of fluorescent material of intermediate molecular mass, that is with a molecular mass between that of the starting HEC and the fluorescent acceptor substrate XGO-SR indicates that the enzyme acts in an essentially stochastic manner. Conversely, when the HEC is tagged at its reducing terminus with the fluorescent XGO-SR, the absence of fluorescent products of intermediate sizes (FIG. 3B) indicates that the enzyme has a preference for binding and cleaving at the xylosylated XGO-SR tag, which is positioned at the reducing end of the HEC:XGO-SR conjugate. It should also be noted that during chemical modification of cellulose with hydroxyethylene groups, the HEC product is likely to be substituted primarily on the more reactive C-6 hydroxyl groups, perhaps in a block-wise fashion. If this were the case, the HEC substrate might represent a structural analog of xyloglucan. However, the barley (1,3;1,4)-β-D-glucan clearly acts as a donor substrate and cello-oligosaccharides act as acceptor substrates.

The substrate specificity of XET enzymes, which involves cleaving a (1,4)-β-D-glucosyl linkage in the donor substrate before transfer to the non-reducing end of the acceptor substrate, would suggest that the HvXET5 re-forms a (1,4)-β-linkage between the reducing end glucosyl residue of the donor polysaccharide, whether that be the HEC or the barley (1,3;1,4)-β-D-glucan, and the non-reducing end of the XXXG-SR acceptor substrate. It is considered unlikely that the polymeric donor molecules would be attached to the xylosyl residues of the XXXG-SR acceptor substrate.

The rate of the reaction catalyzed by the HvXET5 enzyme described here with HEC is comparable with that on TXG (see Table 2). Values for the Kₘ and k_{cat} constants with TXG were 3 mg·ml⁻¹ and 1·10⁻⁷ s⁻¹, respectively, and for the acceptor substrate XXXGol the values of Kₘ and k_{cat} were 69·10⁻⁶ M and 1.5·10⁻⁷ s⁻¹, respectively. The rate of the reaction with (1,3;1,4)-β-D-glucan is relatively slow (see Table 2), but it would be anticipated that contact between a large molecular mass donor-enzyme complex and the non-reducing terminus of the acceptor substrate might not occur quickly.

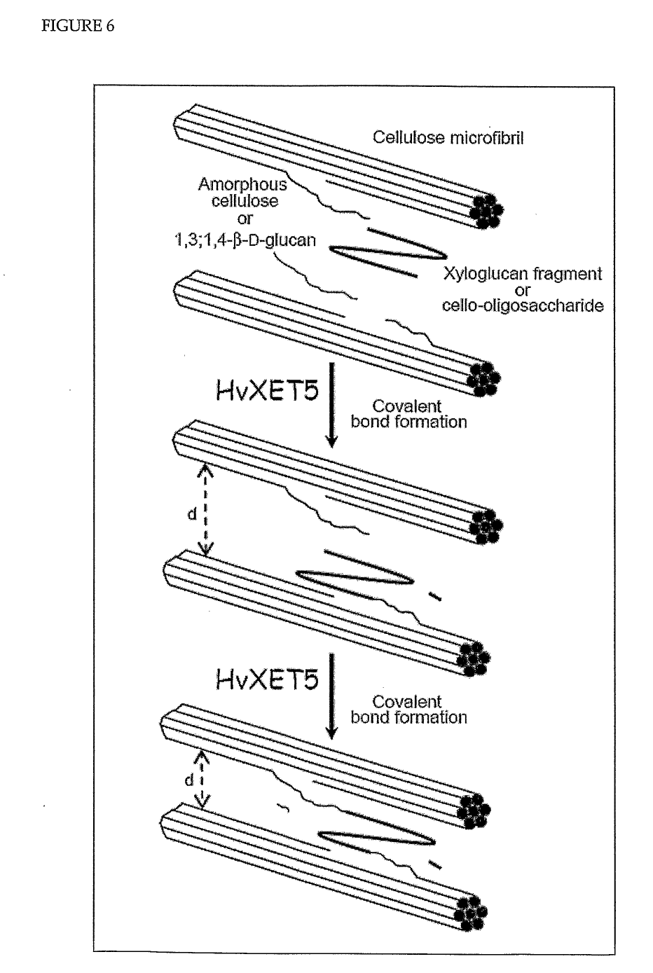

In vivo, cellulose, (1,3;1,4)-β-D-glucans and xyloglucans in plant cell walls could be linked by XTH enzymes (such as HvXET5) to create a very large, continuous molecular network within the wall and would significantly alter the strength of walls, their porosity and flexibility (see FIG. 6). Potential accessibility and diffusion limitations in the cell wall environment could greatly reduce the catalytic rates in muro. The cell might compensate for this through the synthesis of relatively large amounts of stable enzyme and this would be consistent not only with the high levels of XET mRNA transcripts that are found in plant cells, but also with the long term stability of the HvXET5 observed here.

Emerging information on the re-modeling of fungal cell walls during spore formation and under stress indicate that GPI-anchored transferase enzymes, some of which are members of family GH16, might also be involved in linking different polysaccharides such as β-D-glucans and chitin in the wall. There are also indications that pectic polysaccharides might be covalently linked with xyloglucans in plant cell walls. However, the purified HvXET5 enzyme did not link polygalacturonan or β-D-galactans to xyloglucan, nor did the HvXET5 enzyme link arabinoxylans to xyloglucans, despite suggestions based on molecular modeling that this was a possibility. However, there are multiple isoforms of XETs in plant cells and it remains possible that other isoforms might prefer different donor and acceptor substrate specificities.

Covalent linkages between different polysaccharides would have important implications for wall rigidity, strength and porosity. A thorough understanding of covalent linkages between wall polysaccharides would also provide opportunities to genetically manipulate agro-industrial processes such as paper production, food quality and texture, malting and brewing, bioethanol production, dietary fibre and ruminant digestibility.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it must be noted that, as used herein, the singular forms “a”, “an” and “the” include plural aspects unless the context already dictates otherwise. Thus, for example, reference to “a polysaccharide transferase” includes a single polysaccharide transferase as well as two or more polysaccharide transferase; “a donor (or acceptor) polysaccharide” includes a polysaccharide cell as well as two or more polysaccharides; and so forth.

1. An isolated or substantially purified polysaccharide transferase, wherein said polysaccharide transferase is capable of catalyzing the formation of a covalent bond between a donor polysaccharide and an acceptor polysaccharide; or a functionally active fragment or variant of said polysaccharide transferase;
wherein said polysaccharide transferase or functionally active fragment or variant thereof is purified to a substantially homogenous form.

2. The polysaccharide transferase of claim 1 wherein at least one of said donor polysaccharide and said acceptor polysaccharide is a polysaccharide other than a xyloglucan; and/or
said donor polysaccharide and said acceptor polysaccharide are of a different type.

3-6. (canceled)

7. The polysaccharide transferase of claim 1 wherein said donor polysaccharide comprises a glucose polymer (other than a xyloglucan) which comprises at least one β(1-4) linkage between two glucose monomers and said acceptor polysaccharide comprises a xyloglucan; or
said donor polysaccharide comprises a xyloglucan and said acceptor polysaccharide comprises a glucose polymer (other than a xyloglucan) which comprises at least one β(1-4) linkage between two glucose monomers.

8-13. (canceled)

14. The polysaccharide transferase of claim 1 wherein said polysaccharide transferase comprises:
(i) the amino acid sequence set forth in one or more of SEQ ID NO: 1, SEQ ID NO:2 or SEQ ID NO:3; or
(ii) an amino acid sequence which encodes a functionally active fragment or variant of polysaccharide transferase referred to at (i).

15-18. (canceled)

19. The polysaccharide transferase of claim 1 wherein said polysaccharide transferase is isolated or substantially purified from a plant, or a part, organ, tissue or cell thereof.

20-24. (canceled)

25. A method for isolating or substantially purifying a polysaccharide transferase or functionally active fragment or variant thereof according to claim 1 from a sample, the method comprising the steps of:
(i) a salt fractionation step;
(ii) an ion-exchange chromatography step;
(iii) a hydrophobic-interaction chromatography step;
(iv) a chromatofocussing chromatography step; and
(v) a size-exclusion chromatography step;
wherein a fraction having polysaccharide transferase specific activity from one step is used in a subsequent step.

26. The method of claim 25 wherein said sample comprises one or more plant cells, a homogenate thereof, or one or more plant cell walls.

27. A method for forming a covalent bond between a donor polysaccharide and an acceptor polysaccharide, the method comprising contacting said donor polysaccharide and said acceptor polysaccharide with a polysaccharide transferase or functionally active fragment or variant thereof according to claim 1.

28. The method of claim 27 wherein at least one of said donor polysaccharide and said acceptor polysaccharide is a polysaccharide other than a xyloglucan; and/or said donor polysaccharide and said acceptor polysaccharide are of a different type.

29-31. (canceled)

32. The method of claim 27, wherein said donor polysaccharide comprises a glucose polymer (other than a xyloglucan) which comprises at least one β(1-4) linkage between two glucyose monomers and said acceptor polysaccharide comprises a xyloglucan; or
said donor polysaccharide comprises a xyloglucan and said acceptor polysaccharide comprises a glucose polymer (other than a xyloglucan which comprises at least one β(1-4) linkage between two glucose monomers.

33-39. (canceled)

40. The method of claim 27 wherein said covalent bond is formed in vitro.

41. An isolated polysaccharide of the structure:
A−B
wherein A and B are polysaccharides which are covalently linked to each other; and wherein said polysaccharide is produced according to the method of claim 27.

42. A method for modulating the extent of covalent bonding between a donor polysaccharide and an acceptor polysaccharide in a cell; the method comprising modulating the level or activity of either a polysaccharide transferase which is capable of catalyzing the formation of a covalent bond between a donor polysaccharide and an acceptor polysaccharide, or a functionally active fragment or variant of said polysaccharide transferase, in said cell.

43. The method of claim 42 wherein at least one of said donor polysaccharide and said acceptor polysaccharide is a polysaccharide is a polysaccharide other than a xyloglucan; and/or
said donor polysaccharide and said acceptor polysaccharide are of a different type.

44-46. (canceled)

47. The method of claim 42, wherein said donor polysaccharide comprises a glucose polymer (other than a xyloglucan) which comprises at least one β(1-4) linkage between two glucose monomers and said acceptor polysaccharide comprises a xyloglucan; or
said donor polysaccharide comprises a xyloglucan and said acceptor polysaccharide comprises a glucose polymer (other than a xyloglucan) which comprises at least one β(1-4) linkage between two glucose monomers.

48-54. (canceled)

55. The method of claim 42 wherein said polysaccharide transferase comprises:
(i) the amino acid sequence set forth in one or more of SEQ ID NO: 1, SEQ ID NO:2 or SEQ ID NO:3; or
(ii) an amino acid sequence which encodes a functionally active fragment or variant of the polysaccharide transferase referred to at (i).

56-57. (canceled)

58. The method of claim 42 wherein the cell is a plant cell.

59. The method of claim 58 wherein the extent of covalent bonding between said donor polysaccharide and said acceptor polysaccharide is modulated in the cell wall of said plant cell.

60-61. (canceled)
